(19) United States
(12) Patent Application Publication
(10) Pub. No.: US 2007/0269843 A1
(43) Pub. Date: Nov. 22, 2007

(54) Diagnostic Assays That Use Mycobacteriophages

(75) Inventor: Charles G. Thornton, Gaithersburg, MD (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Integrated Research Technology, LLC.

(21) Appl. No.: 11/579,087

(22) PCT Filed: Apr. 25, 2005
(86) PCT No.: PCT/US05/14098

§ 371 Date: Jun. 20, 2007

Related U.S. Application Data
(60) Provisional application No. 60/565,848, filed on Apr. 28, 2004.

(51) Int. Cl. C12Q 1/04; C12Q 1/24; C12Q 1/34

(52) U.S. Cl. 435/18; 435/30; 435/34

(57) ABSTRACT

The present invention provides a method for a rapid and efficient mycobacteriophage-based diagnostic assay for the presence of microbacteria that have mycolic-acid structures in their outer membranes, such as mycobacteria, using betaine-like detergents.

### FIELD OF THE INVENTION

The present invention is in the area of microbiological sample processing. Specifically, the present invention is directed to methods for improving the utility and performance of diagnostic assays that use mycobacteriophage to determine the presence of microorganisms that contain mycolic acid structures in their outer membranes in specimens being processed for clinical analysis. The present invention thus facilitates detection of microorganisms that contain mycolic acid structures in their outer membranes in clinical samples.

### BACKGROUND OF THE INVENTION

Mycobacteriophage are bacteriophages that specifically infect mycobacteria. Such phages can be either lysogenic (i.e., virulent, causing efficient and complete lysis of the infected host), or temperate (i.e., do not cause lysis, but permit the bacillus to exist in a chronically infected state). Lysogenic strains of bacteriophages have been used to develop “plaque assays.” The principle underlying such plaque assays is that when a bacterium infected with a lysogenic bacteriophage is plated on a Petri dish with an uninfected bacterium (e.g., a helper strain) that is also susceptible to infection by the same phage, small clearings (or plaques) will form where the original bacterium resided as a result of infection and lysis of the helper strain. Diagnostic assays have been developed on this principle, and are based on the specificity of such bacteriophages to infect specific hosts. By treating clinical samples suspected of harboring a Mycobacterium (e.g., Mycobacterium tuberculosis) with a lysogenic strain of mycobacteriophage, removing excess phage, and then plating such treated samples with the appropriate helper cells (e.g., Mycobacterium smegmatis), the occurrence of plaques are diagnostic for the presence of Mycobacterium in the original clinical sample.

Indeed, several diagnostic assays are currently available that use such mycobacteriophages to detect the presence of M. tuberculosis in respiratory specimens. Tuberculosis (i.e., infections caused by M. tuberculosis) is the most prevalent infectious disease in the world today, infecting approximately one-third of the world's population, and killing more people worldwide than any other single pathogen. The vast majority of these cases are in developing countries where resources are severely limited. Hence, there is a need for a rapid and inexpensive diagnostic assay to identify infected individuals quickly and accurately. Mycobacteriophage assays offer such promise.

Diagnostic assays that use mycobacteriophage to detect the presence of mycobacteria in biological and inorganic samples must first be processed to prepare such samples for said assays. Current methods of processing biological and inorganic samples suspected of containing one or more mycobacteria, for the detection of such mycobacteria by mycobacteriophage-based assays, are constrained by the harshness of these methods. For example, the contemporary methods are based primarily on the utilization of caustic acids and alkalis, such as sodium hydroxide (NaOH), sulfuric acid, and oxalic acid. Such specimen processing methods are necessary to decontaminate clinical specimens to alleviate contaminating pathogenic and saprophytic microorganisms that would interfere with culturing of mycobacteria; however, such methods also kill as much as 90% of the mycobacteria present, and more importantly, remove receptors from the surface of the few remaining bacilli that are essential for the infection of such mycobacteria by these mycobacteriophages. Consequently, additional preparative steps following the standard specimen processing methods are required prior to such plaque assays. For example, processed sediments must be washed to remove excess acids or alkalis. Such wash step(s) necessitate that the processed specimen be subjected to centrifugation a second time—an extremely labor intensive procedure. In addition, mycobacteria that have been treated with such acids or alkalis must be allowed to recuperate prior to infection: bacilli must be cultured in nutrient broth to recover from exposure to caustic agents and to restore the requisite receptors that permit infection by mycobacteriophage. This significantly lengthens the time needed to obtain the final result. Hence a need exists for a method that facilitates recovery of a desired mycobacteria from a sample that does not need to be washed and/or cultured prior to testing when using a mycobacteriophage plaque assay for diagnostic purposes.

Procedures designed to reduce the influence of specimen processing methods on diagnostic utility would further improve the ability to correctly diagnose infections caused by bacteria containing mycolic acid structures, especially the diagnosis of mycobacterial infections, and most specially infections caused by Mycobacterium tuberculosis.

### SUMMARY OF THE INVENTION

In an effort to find a more efficient method for preparing biological and inorganic samples for the detection of mycobacteria by mycobacteriophage assays, the inventor evaluated interfacing a mycobacteriophage assay with methods for processing clinical samples with the “betaine-like” detergents of U.S. Pat. No. 5,658,749, U.S. Pat. No. 6,004,771, and WO 95/27076. These studies resulted in the discovery of methods and compositions for preparing extracts of biological and inorganic samples that allow such samples to be more efficiently prepared for detection of the presence of microorganisms that contain mycolic-acid structures in their membranes, and especially mycobacteria, by use of in mycobacteriophage assays. The compositions and methods of the invention preclude the need for washing processed sediments to remove caustic acids and alkalis, resulting in a significant savings in labor associated with performing such assays. The compositions and methods of the invention also retain the viability of microorganisms that contain mycolic acid like structures in their outer membranes to a degree that eliminates completely the need to culture such microorganisms prior to detection in said mycobacteriophage assays. This significantly decreases the time necessary to obtain a result, as compared to the mycobacteriophage assays in the art. As a result of eliminating the need to pre-culture samples prior to assay, the compositions and methods of the invention reduces, or completely eliminates, the need to incorporate antibiotics in the media used to propagate the helper strain, thereby further reducing the expense associated with performing said mycobacteriophage assays. These compositions and methods are especially useful for the processing of samples for the detection of mycobacteria using mycobacteriophage plaque assays.

### BRIEF DESCRIPTION OF THE DRAWINGS

FIG. 1: The experimental procedure used to evaluate the consequence of exposure of Mycobacterium tuberculosis ATCC 27294 to CB-18 in the context of a mycobacteriophage assay is shown.

FIG. 2: The experimental procedure used to evaluate the effect of the presence of CB-18 on Mycobacterium tuberculosis ATCC 27294 during infection by the mycobacteriophage D29 is shown.

FIG. 3: The experimental procedure used to evaluate the effect of combining exposure to CB-18, and carrying CB-18 into the infection buffer in the context of a mycobacteriophage assay is shown.

FIG. 4: The experimental procedure used to evaluate the consequence of exposure of Mycobacterium tuberculosis ATCC 27294 to lytic enzymes before infection in the context of a mycobacteriophage assay is shown.

### DETAILED DESCRIPTION OF THE PREFERRED EMBODIMENTS

The invention described herein uses “betaine-like” detergents as described in U.S. Pat. No. 5,658,749, U.S. Pat. No. 6,004,771, and WO 95/27076 to process clinical samples. In the methods of the invention, there is no need to wash the sediment or pellet to remove undesirable caustic agents, and there is no need to culture (pre-culture) the sample containing the mycobacteria prior to a desired plaque assay. By avoiding such pre-culturing, complications of the plaque assay associated with contamination during lawn development with helper cells are reduced or avoided altogether, thereby obviating the need for antibiotics in the assay. In addition, the invention has significant advantages in that the methods greatly reduce the labor required to prepare such samples by eliminating the need to wash processed sediments

In the description that follows, a number of terms used in the chemical arts and in microbiological processing are extensively utilized. In order to provide a clear and consistent understanding of the specification and claims, including the scope to be given such terms, the following definitions are provided.

By “mycobacteriophage” is meant a virus that specifically infects microorganisms of the genus Mycobacterium, as understood in the art (Hatfull, et al., Mycobacteriophages: Cornerstones of Mycobacterial Research. In: Bloom, B. R., ed. Tuberculosis: Pathogenesis, Protection and Control. Washington, D.C., American Society for Microbiology Press, (1994) pp. 165-183; Sarkis, et al. Methods Mol. Biol. 101:145 (1998); Hatfull, G. F., Mycobacteriophages. In: Ratledge, C. and Dale, J., eds. Mycobacteria, Molecular Biology and Virulence. London, Blackwell Science Ltd., (1999) pp. 38-58; Hatfull, G. F., Molecular Genetics of Mycobacteriophages. In: Hatfull, G. F. and Jacobs, W. R., eds. Molecular Genetics of Mycobacteria. Washington, D.C., American Society for Microbiology Press, (2000) pp. 37-54, all incorporated herein by reference). Over 250 mycobacteriophages have been identified to date (McNerney, R. Int. Jour. Tuberc. Lung Dis. 3:179 (1999)). The best characterized mycobacteriophages include “L1”, “L5”, “D29”, “TM4”, “Bxb1”, “DS6A”, and “13”, as well as those that are genetically engineered, such as “phAE40”, “phGS18”, and “phBD8.”

By “plaque assay” is meant a diagnostic assay wherein a mycobacteriophage is mixed with a prepared sample to facilitate infection of the target microorganism (e.g., Mycobacterium tuberculosis) by said mycobacteriophage. After removal of such mycobacteriophage by methods known in the art (e.g., treatment with ferrous ammonium sulfate (McNerney, R., et al., Res. Microbiol. 149:487 (1998)), said sample is then mixed with helper cells and permitted to incubate for a time and at a temperature that facilitates production of a helper cell lawn as understood in the art. The reproduction of phage in the context of the plaque assay results from primary infection in said sample and subsequent infection of said helper cells that form the lawn. Propagation of mycobacteriophage in the helper cell population results in small clearings or plaques as understood in the art. The observation of plaques is said to be confirmatory or diagnostic for the presence of the target microorganism in the clinical sample.

By “helper strain” or “helper cells” is meant a microorganism (e.g., Mycobacterium smegmatis) that is closely related to the target microorganism, and that may be infected by the mycobacteriophage used in the plaque assay. The function of such helper cells is to generate an opaque lawn as understood in the art, such that the occurrence of a primary infection in the form of plaques on said lawn are more easily observed.

By “lysogenic” is meant a strain of mycobacteriophage that is capable of lysing the target microorganism and the helper cells, the latter of which is necessary to produce the characteristic clearings in the plaque assay.

The term “betaine-like” is synonymous with “SB-18-like” as used in WO 95/27076, incorporated herein by reference. Betaine-like detergents according to WO 95/27076 have the ability to disperse cords (and clumps) of mycobacteria and/or compensate buoyancy of the mycobacteria. Dispersion of mycobacteria that cord, such as, for example, Mycobacterium tuberculosis complex (MTB) organisms, facilitates detection by increasing the probability that aliquots taken for detection be representative of all the types of the whole sample. Betaine-like detergents that disperse cords have an alkyl chain length that is greater than 16 carbon atoms, and alkyl chains with 18-20 carbon atoms are most preferred.

Betaine-like detergents also have the ability to facilitate collection of mycobacteria, such as, for example, Mycobacterium avium complex (MAC) organisms, that do not grow in clumps, by compensating, to some degree, the natural buoyancy of such organisms. Such compensation preferably occurs by a mechanism that involves movement of the detergent into the bacterial cell. Betaine-like detergents that compensate buoyancy preferably have an alkyl chain length greater than 12 carbon atoms, and most preferably 16-20 carbon atoms.

Therefore, “betaine-like,” as used herein includes structures as described in Tables 2 and 3 of WO 95/27076, U.S. Pat. No. 5,658,749, and U.S. Pat. No. 6,004,771, all incorporated herein by reference, including, for example, the CB-like, SB-like, HSB-like, PB-like, StB-like, PhB-like, SoB-like, RevB-like, AO-like, cAB-like, and ImB-like compounds that possess SB-18-like activity, as described in WO 95/27076 and in U.S. Pat. No. 5,658,749, and U.S. Pat. No. 6,004,771.

By “betaine-like” is meant a zwitterionic compound of the structure shown in Table 1.

Table 1: The Structure of Alkyl Betaines

The general structure of n-alkyl betaines is shown. R₁ is the hydrophobic alkyl chain, and a is the “linkage” connecting R₁ to the cation, β. R₂ and R₃ modify the cation, when required. R₄ is the “bridge” that connects the cation to the anion, γ.

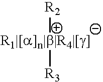

| R₁ | C₈—C₂₂ |
|---|---|
| α | \|CH₂\|, \|CH(OH)\|, \|(CO)—NH—CH₂CH₂CH₂\|, \|O\|, \|C(O)\| |
| n | 0 or 1 |
| β | \|N^{⊕}\|, \|P^{⊕}\|, \|S^{⊕}\| |
| R₂ | \|H, \|CH₃, \|C₂H₅, \|C₃H₇, \|C₄H₉ |
| R₃ | \|H, \|CH₃, \|C₂H₅, \|C₃H₇, \|C₄H₉ |
| R₄ | \|CH₂\|, \|C₂H₄\|, \|C₃H₆\|, \|C₄H₈\|, |
| | \|C₅H₁₀\|, \|C₆H₁₂\|, \|CH₂\|C₆H₄\|, \|CₘH₂ₘ\|, |
| | \|CH(OH)CH₂CH₂\|, \|CH₂CH(OH)CH₂\|, \|CₘH₂ₘ₋₁(OH)\|; |
| | where m ≧ 1 |
| γ | —SO₃^{⊖}, —OSO₃^{⊖}, —COO^{⊖}, —OPO₃^{⊖}, —PO₃^{⊖}, |
| | —PO₂^{⊖}— |

By “CB-like” is meant those betaine-like detergents having a carboxylate (—COO^{⊖}) moiety as the anion (e.g., carboxybetaine-like). By “SB-like” is meant those betaine-like detergents having a sulfonate (—SO₃^{⊖}) moiety as the anion (e.g., sulfobetaine-like). By “HSB-like” is meant those betaine-like detergents having a sulfonate moiety as the anion, and a hydroxyl group (—OH) in the bridge (e.g., hydroxysulfobetaine-like). By “PB-like” is meant those betaine-like detergents having either a phosphate (—OPO₃^{⊖}), phosphonate (—PO₃^{⊖}), or a phosphinate (—PO₂^{⊖}) moiety as the anion (e.g., phosphobetaine-like). By “StB-like” is meant those betaine-like detergents having a sulfate (—OSO₃^{⊖}) moiety as the anion (e.g., sulfatobetaine-like). By “AO-like” is meant those betaine-like detergents having an oxide radical (—O^{⊖}) as the anion (e.g., amine oxide-like). By “PhB-like” is meant those betaine-like detergents having a phosphonium (—P^{⊕}—) moiety as the cation (e.g., phosphoniumbetaine-like). By “SoB-like” is meant those betaine-like detergents having a sulphonium (—S^{⊕}—) moiety as the cation (e.g., sulphoniumbetaine-like). By “n-alkyl betaine” is meant those betaine-like detergents having an ammonium (—N^{⊕}—) moiety as the cation (e.g., n-alkyl betaine-like). By “ImB-like” is meant those betaine-like detergents having a imidazolinium moiety as the cation (e.g., imidazoliniumbetaine-like). By “RevB-like” is meant those betaine-like detergents wherein the alkyl chain is covalently attached to the anion, as opposed to the cation (e.g., reverse betaine-like). By “cAB-like” is meant those betaine-like detergents wherein the alkyl chain is covalently attached to the bridge, as opposed to either the cation or the anion (e.g., c-alkyl betaine-like).

By “CB-18” is meant N-(3-carboxypropyl)-N,N-dimethyl-1-octadecanaminium, inner salt. CB-18 is also known as N,N-dimethyl-N-(n-octadecyl)-N-(3-carboxypropyl) ammonium inner salt, or C₁₈-carboxypropylbetaine. CB-18 has been assigned the CAS®No. 78195-27-4.

By “SB-18” is meant N-octadecyl-N,N-dimethyl-3-ammonio-1-propane-sulfonate (CAS®No. 13177-41-8).

By “SB-16” is meant N-hexadecyl-N,N-dimethyl-3-ammonio-1-propane-sulfonate (CAS®No. 2281-11-0).

By “SB-14” is meant N-tetradecyl-N,N-dimethyl-3-ammonio-1-propane-sulfonate (CAS®No. 14933-09-6), and by “SB-12” is meant N-dodecyldecyl-N,N-dimethyl-3-ammonio-1-propane-sulfonate (CAS®No. 14933-08-5).

By “mycolic acid structures” is meant chemical compounds that can be described as a β-hydroxy acid substituted at the α-position with a moderately long aliphatic chain, as understood in the art (Goren, M. B. Bact. Rev. 36:33-64 (1966), incorporated herein by reference). The term is synonymous with “mycolic acid-like structures.” Mycolic acid structures are also collectively termed “mycolic acids.” Additional tables of representative mycolic acid structures, including some that are unsaturated, cyclopropanoid, methoxylated and ketonic acids, may also be found, for example, in Lederer, E. Chem. Phys, Lipids 1:294-315 (1967); Lederer, E. Pure Appl. Chem. 25:135-165 (1971), both incorporated herein by reference. Mycolic acid structures are acid-stable molecules. Examples of classes of microorganisms that contain mycolic acid structures in their outer membranes would be Mycobacterium, Nocardia, Corynebacterium, and Rhodococcus, as understood in the art.

By “Good buffer” is meant an aqueous solution containing a chemical compound that resists pH changes as understood in the art (Beynon, R. J., et al., Buffer Solutions, The Basics. IRL Press, New York (1996), incorporated herein by reference). Such buffers serve to stabilize the hydronium ion concentration of aqueous solutions that are used in the biological, biomedical and biochemical arts. There are numerous Good buffers useful in the methods and compositions of the invention. Such Good buffers are named more because of the original description of a series of buffers by Good, N. E., et al., Biochemistry 5:467-477 (1966) (incorporated herein by reference) that were considered to possess attractive qualities for use in the biological, biomedical and biochemical arts, than for the fact that they were “good” choices for use in the biological, biomedical and biochemical arts. Examples of Good buffers useful in the methods of the invention include those that are carboxylic acid-based, alcohol-substituted amines, and sulfonic acid-based, and especially those sulfonic acids that are alcohol-substituted, cyclohexyl-substituted, morpholino-substituted, and piperazine-substituted.

Unless otherwise defined, by “contaminant,” is meant a living or detectable microorganism, for example, a bacterium, a fungus or mold, or yeast, as understood in the art (Manual of Clinical Microbiology 6^{th} Edition, Murray, P. R. et al., eds. ASM Press, Washington, D.C. (1995), incorporated herein by reference), that is present at a detectable level in a preparation and is other than a desired microorganism that is of interest that contain mycolic acid structures in its outer membrane.

By “lytic enzyme” is meant an enzyme, as understood in the art, and as described in U.S. Pat. No. 5,985,593 (incorporated herein by reference), that has enzymatic activity against the components of the outer membrane, cell wall, capsid or capsular structures of contaminating microorganisms. That is to say that the substrates of such lytic enzymes are present in the components of the outer membrane of such contaminants. Lytic enzymes are said to have “lytic activity.” Such lytic activity in the methods of the invention serves to destabilize the structural integrity of such contaminant. For example, since the outer membrane is an essential aspect of structural integrity and/or viability of the contaminant, destabilizing said outer membrane matrices causes an inherent change in the ability of the contaminants to remain physically intact, and/or to continue to survive (e.g., maintain viability).

By “treatment” or “treating” is meant to incubate specimens with, expose specimens to, or otherwise cause the specimen to come in contact with enzymes, proteins, chemicals, or inert substrates under conditions that serve to reduce the complex nature of specimens (e.g., to liquefy specimens, solubilize components, and/or remove inhibitors). Treatment of specimens with enzymes such as for example, proteases, glycosidases, and/or DNase's serves to cleave, degrade or digest proteins, polysaccharides, and/or DNA, respectively, in the specimen matrix as understood in the art (i.e., “enzymatic digestion”) to liquefy specimens and solubilize such digested components. Treatment with reducing agents such as dithiotheritol (DTT), N-acetyl-L-cysteine (NALC) or β-mercaptoethanol (BME) serves to reduce disulfide bonds as understood in the art, and acid or alkaline treatments serve to denature and/or solubilize components of the specimen matrix, thereby reducing the complexity of said matrix as understood in the art (i.e., “chemical digestion” of the specimen). Exposing specimens to inert substrates in the form of beads or fibers can also serve to reduce the complexity of specimens. Cross linked polymers such as Sephadex®, cellulose, or ion exchange resins, as understood in the art, can be used to either remove components of the specimen matrix by chromatographic means, or clarify the specimen matrix by physically separating soluble from insoluble components, or both (i.e., “purify” the specimen).

By “specimen” is meant a material from which a sample can be obtained for a desired analysis. Specimens include, but are not be limited to, biological samples and inorganic samples.

By “biological sample” is meant a sample derived from, or taken from, a specimen of biological origin, such as a specimen taken from an animal (including human) or plant. Biological samples can be derived from, or taken from any part of the biological organism. Biological samples include, but are not limited to any biological liquids and solids, for example, expectorated matter (for example, sputum, saliva and phlegm), bronchial lavages and analogous respiratory washings, feces, tissue samples including skin samples, gastric aspirates, urine, tears, perspiration, blood and cerebral spinal fluid (CFS). Any animal species may be used as a source for such samples, including but not limited to ruminant animals (such as members of the bovine family (bulls, ox, buffalo, cattle, cows, etc.) or members of the ovine family (sheep, etc.)), pigs, fish, members of the avian family (birds), badgers, deer, elk, cats and dogs. The term biological sample is also intended to include a specimen taken from a processed or an unprocessed food source. Such processed or unprocessed food sources include, for example, a specimen derived from meat, diary products (especially, for example, eggs, cheese and milk), plants and processed food derived from plants. Food sources also include animal feed, for example, cattle feed, silage, hay, alfalfa bales, and food samples from pastures. The term biological sample is also intended to include specimens taken from a cell culture source (such as monocyte or fibroblast cultures).

By “inorganic sample” is meant a sample derived from, or taken from, a non-biological specimen, such as, for example, from an environmental source such as soil, mud, sludge, water, sawdust and air.

By a sample that is “derived from” a specimen or extract thereof is meant a sample that is directly taken from or otherwise indirectly prepared from such specimen or extract thereof.

By “antibiotic” is meant a compound that has a deleterious effect on the viability, integrity, or competence of a contaminant, as understood in the art (see: Murray, P. R. et al., eds. Manual of Clinical Microbiology, ASM Press, Washington, D.C. (1995) pp. 1281-1307, 1385-1404, and 1405-1414; Kucers, A. et al., The Use of Antibiotics 4^{th} ed. J. B. Lippincott Co. Philadelphia, Pa. (1987); and Lorian, V. ed. Antibiotics in Laboratory Medicine 2^{nd} Edition, Williams & Wilkins, Baltimore, Md., all incorporated herein by reference). The term antibiotic is synonymous with “antimicrobial,” as used herein.

According to the invention, a sample or clinical specimen that is suspected of harboring a microorganism that contains mycolic acid structures, especially the mycobacteria, is first processed with any of the methods of U.S. Pat. No. 5,658,749, U.S. Pat. No. 6,004,771, and WO 95/27076 to the sediment or button (pellet) stage. The resulting sediment or button is then “pre-cultured,” that is, it is resuspended in nutrient broth, such as, for example 7H9 media, and then allowed to incubate for a time and at a temperature that will allow such microorganisms, and especially mycobacteria, to initiate the cellular processes associated with replication, or allow the mycobacteria to replicate. The resulting broth is then analyzed for the presence of such microorganisms, and especially, mycobacteria, by any mycobacteriophage-based plaque assay. In this embodiment washing of the processed sediment is avoided.

In a more preferred embodiment the sample or clinical specimen that is suspected of harboring a microorganism that contains mycolic acid structures, and especially, mycobacteria, is processed with any of the betaine-like detergents to the sediment or button (pellet) stage. The resulting sediment or button is then resuspended in nutrient broth, but NOT allowed to incubate for a time and at a temperature that will allow the mycobacteria to initiate the cellular processes associated with replication, or allow the mycobacteria to replicate. The resulting broth is then analyzed for the presence of such microorganisms, and especially, mycobacteria, by any mycobacteriophage-based plaque assay. In this embodiment both washing of the processed sediment is avoided, and the pre-culture step is avoided.

In the most preferred embodiment the sample or clinical specimen that is suspected of harboring a microorganism that contains mycolic acid structures, and especially, a mycobacteria, is processed with any of the methods taught in U.S. Pat. No. 5,658,749, U.S. Pat. No. 6,004,771, and WO 95/27076 to the sediment or button (pellet) stage. The resulting sediment or button is then resuspended in a buffer, preferably a Good buffer. No washing of the sediment or pre-culture of the processed sample is performed. The resulting buffer suspension is then analyzed by any mycobacteriophage-based plaque assay. In this embodiment, washing and pre-culture of the processed sediment are both eliminated; however, the resuspension buffer that is chosen in this embodiment is such that its composition has been optimized for use in conjunction with the mycobacteriophage-based assay used to analyze the sediment. While the buffer used to process such samples with any of the betaine-like detergents does not necessarily have to be the same buffer used to resuspend the button, in a variation of this embodiment the buffer used to process such samples with any of the betaine-like detergents is the same as the buffer used to resuspend the button. In this variation, divalent metal cations, such as Mn⁺², Mg⁺², and/or Ca⁺² may be used to supplement the resuspension buffer to further facilitate infection by a desired mycobacteriophage. This is the most preferred embodiment because it is the least labor intensive, generates the clinical diagnostic result in the shortest period of time, reduces the quantity of broth required (i.e., reduces the cost of the assay), and reduces the dependence of the assay on antibiotics (i.e., further reducing the cost of the assay) because contaminants are not given the opportunity to replicate prior to any mycobacteriophage-based plaque assay, and the helper cells (e.g., M. smegmatis) can more efficiently compete for nutrients in the culture media.

In any of the embodiments above lytic enzymes can be used to treat samples or processed sediments as taught in U.S. Pat. No. 5,985,593. Such lytic enzyme treatment can be performed after processing with any of the betaine-like detergents, but before infection with any of the mycobacteriophage assays. Alternatively, lytic enzyme treatment can be performed after both processing with a betaine-like detergent and the infection with any mycobacteriophage, but before plating with helper cells. The purpose of treating with the lytic enzymes of U.S. Pat. No. 5,985,593 is to reduce the complexity of the specimen in regard to contaminating microorganisms, thereby reducing complications associated with contamination during pre-culture or helper cell lawn development.

In any of the embodiments above treatment of specimens with other chemicals, enzymes, proteins, cellulose, and/or cross linked polymers in the form of beads or fibers can be used to reduce the complex matrix of the specimen itself. Such treatment(s) can be performed either before or after processing with any of the betaine-like detergents, but before infection with any of the mycobacteriophage assays. Such treatments serve to degrade or denature proteins (e.g., actin, fibrin, and immune complexes), glycoprotein's (e.g., mucins), or nucleic acids (e.g., DNA) present in the entangled mesh that comprises clinical specimens (e.g., the mucus present in respiratory specimens), or reduce the amount of insoluble material present in clinical specimens by solubilization or clarification. Such treatments can take place before or after processing with any of the betaine-like detergents, but before infection with any of the mycobacteriophage assays. Alternatively, such treatments can be performed simultaneously with processing with a betaine-like detergent.

None of the three preferred embodiments described above is intended to be exclusive to the other.

The first mycobacteriophage was identified almost 60 years ago (Gardner, G. M., et al. Proc. Soc. Exper. Biol. Med. 66:205 (1947)). Since then, more than 250 bacteriophages that specifically infect the genus Mycobacterium have been identified (Sarkis, et al., Methods Mol. Biol. 101:145 (1998)). The utility of mycobacteriophage in mycobacterial research is firmly established, and these phages have been examined for both diagnostic and therapeutic use (Hatfull, et al., Mycobacteriophages: Cornerstones of Mycobacterial Research. In: Bloom, B. R., ed. Tuberculosis: Pathogenesis, Protection and Control. Washington, D.C., American Society for Microbiology, (1994) pp. 165-183; McNerney, R. Int. Jour. Tuberc. Lung Dis. 3:179 (1999)). Their use in diagnostics in a classic plaque assay format has been commercialized as both a primary screening test to identify individuals with active tuberculosis, as well as in drug susceptibility testing (Albert, H., et al., Int. Jour. Tuberc. Lung Dis. 6:523 (2002); Albert, H., et al., Int. Jour. Tuberc. Lung Dis. 6:529 (2002); Albert, H., et al., Int. Jour. Tuberc. Lung Dis. 7:284 (2003); Albay, A., et al., Diag. Microbiol. Infect. Dis. 46:211 (2003)). Mycobacteriophages have also been genetically engineered to include reporter genes such as luciferase or β-galactosidase to develop chemiluminescent and colorimetric plaque assays, respectively (WO 93/16172; WO 94/25572; U.S. Pat. No. 5,968,733; U.S. Pat. No. 6,225,066; U.S. Pat. No. 6,300,061).

Current diagnostic methods in tuberculosis rely on smear microscopy, culture, nucleic acid amplification, and/or serological methods. Smear microscopy is rapid and inexpensive, but lacks sensitivity and specificity (Burdash, N. M., et al., Jour. Clin. Microbiol. 4:190-191 (1976); Aber, V. R., et al., Tubercle. 61:123-133 (1980); Lipsky, B. A., et al., Rev. Infect. Dis. 6:214-222 (1984); Murray, P. R., et al., Ann. Intern. Med. 92:512-513 (1980)). Whereas culture is the current gold standard for identifying active disease, due to the slow growth of M. tuberculosis, cultures must be held for up to eight weeks before they can be reported as negative. Nucleic acid amplification was predicted to hold the greatest promise with respect to providing the most rapid and sensitive technique, but prior to the invention described in WO 95/27076, this promise has not materialized due to the technical expertise required to perform these tests, their excessive cost, the nature of the disease, and the vulnerability of these assays to inhibition: sensitivities of approximately 50% have been reported among smear negative and culture positive specimens (Catanzaro, A., et al., Am. J. Respir. Crit. Care Med. 155:1804-1814 (1997)). Serological methods such as skin testing (e.g., PPD and Mantoux), are more rapid, but also lack sensitivity and cannot distinguish between latent and active infections, nor can these tests distinguish between individuals vaccinated with BCG or exposed to M. tuberculosis. Therefore, diagnostic assays that utilize mycobacteriophage fill an important niche in tuberculosis testing: these assays can be engineered for a high degree of specificity, identify patients with active disease, and results can be obtained within several days. In addition, these tests require minimal technical expertise and can be performed in developing countries where the tests are needed most for a reasonable cost.

The weakness of these mycobacteriophage assays is the relatively large amount of labor required to perform such assays, and the inefficiencies that arise as a result of the methods used to prepare clinical samples for testing for the presence of microorganisms that contain mycolic acid structures. Current art teaches that samples submitted for testing should be processed with methods recommended by the Centers for Disease Control (CDC) and Prevention (Kent, P. T. et al., “Public Health Mycobacteriology,” in A Guide for the Level III Laboratory, U.S. Department of Health and Human Service, Centers for Disease Control, (1985) pp. 31-46). The most common method for the extraction of organisms with mycolic acid structures, such as mycobacteria from biological and inorganic samples to ascertain the presence of said microorganisms in such samples commonly utilizes the NALC/NaOH method (Kubica, G. P. W. et al., Am. Rev. Resp. Dis. 87: 775-779 (1963)). When organisms with mycolic acid structures are processed with the NALC/NaOH method, the specimen is first mixed with an equal volume of a solution containing 2%-4% NaOH and 0.5% of the reducing agent N-acetyl-L-cysteine (NALC). The purpose of this step is to both decontaminate and liquefy the specimen (especially respiratory specimens). The NALC facilitates liquefaction of the specimen, while the NaOH kills most contaminants, but at the expense of viability: in excess of 90% of the bacilli are killed in this step by exposure to these agents (Yajko, D., et al., Jour. Clin. Microbiol. 33:1944-1947 (1995); Burdz T. V. N., et al., Diagn. Microbiol. Infect. Dis. 47:503-509 (2003)). The specimen is then subjected to centrifugation and the resulting sediment (e.g., pellet or “button”) is used as the source of the sample that is to be assayed for the presence of the desired microorganism that contains mycolic acid structures.

Whereas killing of microorganisms that contain mycolic acid structures produces one inefficiency, another inefficiency arises in the poor recovery of such microorganisms after processing due to the fact that such microorganisms are buoyant (Silverstolpe, L., Nord. Med. 40/48:2220-2222 (1948)). Specifically, the processing methods that utilize NALC/NaOH do not overcome the innate buoyancy of said microorganisms, thereby introducing another inefficiency in said plaque assays. When clinical samples are processed with any of the recommended methods of Kent, P. T. et al., “Public Health Mycobacteriology,” in A Guide for the Level III Laboratory, U.S. Department of Health and Human Service, Centers for Disease Control, (1985) pp. 31-46, additional labor-intensive steps to neutralize and remove caustic agents are also necessary: this is typically accomplished by subjecting the button to a wash step(s) that involves additional centrifugations. The most damaging aspect of such processing methods as it relates directly to performance of mycobacteriophage-based assays; however, is that receptors necessary to infect microorganisms that contain mycolic acid structures with mycobacteriophage are removed (i.e., stripped from the surface of the microorganism, and as such make the very microorganisms that it is desired to infect resistant to infection). Hence, prior to the methods of the invention, in order to assay with a mycobacteriophage assay, such microorganisms must be subjected to a recovery period that involves culture. According to the invention, while microorganisms that contain mycolic acid structures can be incubated in nutrient broth for a time and at a temperature that allows recovery of bacilli and regeneration of such receptors prior to any method that comprises detection of microorganisms with mycolic acid structures by a mycobacteriophage-based plaque assay, it is not required and in a preferred embodiment, such culture or “pre-culture” is not performed.

One significant complication of such a pre-culturing step is that some contaminants also survive sample processing and are allowed to recover during this pre-culture phase as well. The complication arises because these contaminants can grow rapidly, thus destroying the ability to observe the presence of plaques (e.g., overgrowth contamination). Consequently, culture media or nutrient broth used during both the pre-culture phase, and during the development of plaques might also contain antibiotics to minimize such overgrowth by contaminants, thereby causing the loss of critical patient samples. Antibiotics may add a significant expense to each diagnostic assay.

The methods of the invention allow the artisan to eliminate such wash steps and/or pre-culturing of said microorganisms. This is a significant advantage in regard to enabling mycobacteriophage assays to be used as a means for rapid, large-scale screening for tuberculosis. While Jacobs et al. (WO 93/16172; WO 94/25572; U.S. Pat. No. 6,225,066; U.S. Pat. No. 6,300,061) teach that such mycobacteriophage assays can be performed “ . . . either directly or after culture . . . ”, they provide no guidance as to how such mycobacteriophage plaque assays might be accomplished in the absence of culture. To the contrary, Jacobs, et al. (WO 93/16172; WO 94/25572; U.S. Pat. No. 6,225,066; U.S. Pat. No. 6,300,061), and Banaiee, N., et al., Jour. Clin. Microbiol. 39:3883 (2001) teach that clinical specimens are to be processed with NALC-NaOH, and that processed sediments are to be cultured for an extended period (i.e., several days) before detection in a mycobacteriophage assay. Albert, H., et al., Int. Jour. Tuberc. Lung Dis. 6:523 (2002); Albert, H., et al., Int. Jour. Tuberc. Lung Dis. 6:529 (2002); Albert, H., et al., Int. Jour. Tuberc. Lung Dis. 7:284 (2003); Albay, A., et al., Diag. Microbiol. Infect. Dis. 46:211 (2003), teach that clinical specimens are to be processed with NALC-NaOH, and that processed sediments are to be washed in nutrient broth prior to an overnight pre-culture incubation: the mycobacteriophage assay is then performed the next day.

Given that the current art teaches that microorganisms with mycolic acid structures, such as mycobacteria, must be subjected to culture prior to performing any mycobacteriophage-based diagnostic assay, the inventor sought to apply the methods taught in U.S. Pat. No. 5,658,749, U.S. Pat. No. 6,004,771, and WO 95/27076 that use the betaine-like detergents. Practice of these sample processing methods has been shown to provide advantages with respect to improving the ability to diagnose mycobacterioses by smear and nucleic acid amplification, but most especially by culture (Thornton, C. G., et al., Jour. Clin. Microbiol. 36:1996 (1998); Conjeo, B. J., et al., Appl. Env. Microbiol. 64:3099 (1998); Manterola, J. M., et al., Eur. Jour. Clin. Microbiol. Inf. Dis. 22:35 (2003); Thornton, C. G., et al., Jour. Zoo Wildlife Med. 30:11 (1999); Thornton, C. G., et al., Jour. Clin. Microbiol. 40:1783 (2002); Laserson, K. F., et al., 4^{th} World Congress on Tuberculosis, abstract #131, p 79 (2002)). The inventor was surprised to discover that the methods taught in U.S. Pat. No. 5,658,749, U.S. Pat. No. 6,004,771, and WO 95/27076 could be modified in such a way that eliminated the need to culture microorganisms prior to being tested in such mycobacteriophage assays. Indeed, the inventor was further surprised to discover that such betaine-like detergent methods could also be used in such a way as to eliminate the requirement for any secondary wash step, thereby further reducing the time and labor associated with performing mycobacteriophage assays. Such discoveries led the inventor to realize that additional savings were also plausible.

These results were surprising for several reasons. First, Thornton, C. G., et al., Jour. Clin. Microbiol. 36:2004 (1998), and Thornton, C. G., et al., Jour. Clin. Microbiol. 36:3558 (1998) teach that carboxybetaines, specifically C₁₈-carboxypropylbetaine (CB-18), has tuberculocidal activity, that exposure to CB-18 compromises the viability of tuberculous mycobacteria, and that there is a time-dependent killing of such mycobacteria. Example 1 shows that exposure of the M. tuberculosis type strain ATCC 27294 to 1 mM CB-18 for 30, 60, 120, and 180 minutes had no deleterious effects on a D-29 mycobacteriophage plaque assay (this experiment was performed in such a way as to dilute CB-18 to negligible levels prior to infection). Whereas assaying the tuberculocidal activity of CB-18 shows a time dependent loss in viability, propagation of mycobacteriophage in bacilli that have been exposed to CB-18 for up to three hours appears to be unaffected. While an overnight incubation in nutrient broth generated slightly more robust results, these results clearly indicated that the receptors necessary for infection are mostly intact, if not completely intact following exposure to CB-18. In addition, there does not appear to be a need to provide a recovery period that would allow processed microorganisms to recuperate from sample processing. Therefore, processing clinical samples suspected of harboring microorganisms that contain mycolic acid structures, especially mycobacteria, with the methods of U.S. Pat. No. 5,658,749, U.S. Pat. No. 6,004,771, and WO 95/27076, can be practiced in such a manner as to avoid culture prior to any mycobacteriophage assay. While pre-culture in nutrient broth following exposure to CB-18 provided more robust results, it is not clear whether the increase in the number of plaques was due to an in vitro amplification of bacilli during this pre-culture step, or whether this was due to generation/regeneration of receptors among a small population of stationary phase bacilli. Since these bacilli were scraped off solid media, the likelihood that some bacilli were actively dividing is exceptionally good, thereby skewing any comparisons. Regardless, a significant population of bacilli contain receptors and these bacilli can support phage infection and replication following exposure to CB-18. Thus, using the method of the invention, the plaque assay can be performed in such a manner as to avoid any culture step prior to such mycobacteriophage assays (e.g., pre-culture).

The second reason such results were surprising was that Sarkis, et al., Methods Mol. Biol. 101:145 (1998) teach that detergents interfere with the ability to infect mycobacteria. White, A. et al., Am. Rev. Tuberc. 77:134 (1958) teach specifically that Tween 80 interferes with the ability to propagate the mycobacteriophage D-29 in M. tuberculosis at concentrations above 0.0012% (i.e., 1 EM). In Example 2 it is shown that the presence of CB-18 during infection of M. tuberculosis ATCC 27294 at concentrations as high as 20 μg/ml (i.e., 52 μM) does not interfere with the assay. This is further surprising since Thornton, C. G., et al., Jour. Clin. Microbiol. 36:2004-2013 (1998) teach that when CB-18 is present at 20 μg/ml in the culture media, the growth characteristics of the M. tuberculosis type strain ATCC 27294 are significantly affected. These results suggest that washing sediments processed with the methods of U.S. Pat. No. 5,658,749, U.S. Pat. No. 6,004,771, and WO 95/27076 may not be necessary prior to any mycobacteriophage plaque, thereby significantly reducing such a labor intensive step.

Example 3 combines the concepts outlined in Examples 1 and 2: M. tuberculosis can be incubated in the presence of 1 mM CB-18 for 90 minutes and immediately taken for infection so long as the concentration of CB-18 carried into the infection media or buffer is below approximately 40 μg/ml.

The inventor further recognized that because of the physio-chemical flexibility of betaine-like detergents as processing reagents, processing with any of the methods of U.S. Pat. No. 5,658,749, U.S. Pat. No. 6,004,771, and WO 95/27076 can be modified in such a way that the composition of the buffer used to process such specimens is the same buffer used to perform the infection in any such mycobacteriophage plaque assay. For example, the betaine-like detergents can be used in practically any of the Good buffers, within a broad pH range, and at virtually any ionic strength. Therefore, the buffer used to process such clinical samples in preparation for analysis by any such mycobacteriophage plaque assay can be matched in a novel way to optimize both processing of such specimens with any of the betaine-like detergents, and performance of the mycobacteriophage plaque assay. Use of a solution(s) to process clinical samples for detection of microorganisms with mycolic acid structures, especially mycobacteria, that is the same as, or identical (or matched with any desired detection assay—that is, compatible with and useful for a desired detection assay) was hereinbefore unimaginable because such processing methods utilized caustic acids and alkalis. Such caustic reagents would have to be neutralized and/or removed in some fashion prior to performing any diagnostic assay. The methods of the invention allow, for the first time, the processing reagent to be matched in terms of pH and composition, with the needs of the detection assay (i.e., any such mycobacteriophage plaque assay).

As a result of the methods of the invention, a consequence of eliminating pre-culture of microorganisms prior to assay in any mycobacteriophage plaque assay is the reduction of contaminants that can interfere with such plaque assays. The fact that many betaine-like detergents have bactericidal and bacteriostatic activity, in conjunction with the view that pre-culture allows such contaminants to recover to some degree prior to performing said mycobacteriophage plaque assays, results in a reduction in losses associated with contamination. This is consistent with U.S. Pat. No. 6,242,486 (incorporated herein by reference) wherein the bactericidal activity of carboxybetaines was taught, as well as the data of Tsubone et al., Jour. Phar. Sci. 80:441-444 (1991) and Voss et al. Jour. Gen. Microbiol. 48:391-400 (1967) (both incorporated herein by reference) who teach that the betaines have a high degree of bactericidal activity, and this activity can be enhanced by adjusting the pH. Therefore, the reliance of mycobacteriophage plaque assays on antibiotics during plaque formation may be eliminated by these methods; however, if desired, antibiotics can be employed to ensure that contamination is eliminated during such plaque assays.

When using a method other than the NALC-NaOH method, such as the methods that utilize a betaine-like detergent according to WO 95/27076, the specimen can be subjected to enzymatic decontamination with lytic enzymes either before infection with mycobacteriophage, during infection with mycobacteriophage, or following infection with mycobacteriophage. In a preferred embodiment, the lytic enzyme cocktail contains lysozyme or lyticase, and in a highly preferred embodiment, at least both lysozyme and lyticase. In an especially preferred embodiment, the enzyme cocktail contains lytic enzyme-containing extracts of Trichoderma and/or Cytophaga, in addition to lysozyme and/or lyticase, but most especially lytic enzyme-containing extracts of both Trichoderma and Cytophaga in addition to lysozyme and lyticase. Lytic enzyme-containing extracts of Lysobacter may be used in place of Cytophaga extracts, if desired. Thus, in another especially preferred embodiment, the enzyme cocktail contains lytic enzyme-containing extracts of both Trichoderma and Lysobacter in addition to lysozyme. Additionally, lytic enzyme preparations of Micromonospora can be used alone or combined with other lytic enzyme preparations. The lytic enzymes can be either natural or recombinant, and in a purified or unpurified form.

When using methods that employ a betaine-like detergent according to U.S. Pat. No. 5,658,749, U.S. Pat. No. 6,004,771, and WO 95/27076, the specimen can be subjected to treatments with proteins, enzymes, chemicals, and/or inert substrates before infection with mycobacteriophage. Example 5 shows that one of the most significant problems with applying mycobacteriophage assays to clinical specimens is that the level of inhibition encountered is severe. The most common specimens analyzed for diagnosing tuberculosis are respiratory specimens (e.g., sputum, alveolar lavage, bronchial washings, etc.). Mucus is a complex matrix of DNA, cellular debris, and filamentous actin derived from lysed neutrophils and leukocytes, all entangled within a gelatinous matrix of mucin, (Fuloria, M., et al., Respir. Care 45:868-873 (2000)). This matrix must be reduced, degraded, or eliminated in order for the mycobacteriophage assays to perform optimally. Proteins, enzymes, or chemicals which target each of the matrix components would be useful means to reduce the complexity of the specimen matrix. Proteins such as gelsolin have been shown to liquefy sputum by cleaving actin filaments (Vasoncellos, C. A., et al., Science 263:969-971 (1994)). Gelsolin is a protein involved in the rearrangement of the cytoskeleton; other proteins, such as the ADF/cofilin family of proteins (Maciver, S. K., et al., Genome Biol. 3:1-12 (2002)) might also be useful in conjunction with the methods of the invention. The chemical swinholide A (Bubb, M. R., et al., Jour. Biol. Chem. 270:3463-3466 (1995)), a cytotoxin isolated from a marine sponge also cleaves actin filaments, and as such would be expected to be useful in the methods of the invention. Enzymes that digest nucleic acids, such as DNA, might also be used to facilitate liquefaction of sputum. For example, the recombinant form of human DNase I (rhDNase) is currently used to facilitate clearing of secretions in cystic fibrosis patients (Shak, S., et al., Proc. Natl. Acad. Sci. 87:9188-9192 (1990)). Nucleases such as DNase I from human or bovine sources are currently commercially available, and would be expected to be useful in the methods of the invention. The most common way to liquefy sputum involves the use of chemical reducing agents such as DTT, NALC, or BME. Such reagents reduce disulfide bonds between mucins. In addition, hypertonic saline (e.g., 1%-7% NaCl) can also be used to liquefy sputum (Robinson, M., et al., Thorax 52:900-903 (1997)).

Whereas protein, enzyme and/or chemical treatments can be used to facilitate liquefaction of any such sputum matrix, these methods only purify/clarify specimens to the extent that components can be solubilized and remain in the supernatant fraction following centrifugation (i.e., that portion of the processed specimen that is discarded). While such enzyme, protein, and/or chemical treatments are useful, and serve an important role in breaking down the specimen matrix, Example 6 suggests that the most inhibitory component(s) of the specimen matrix are associated with the insoluble, precipitated material that forms the button (pellet). Therefore, methods that serve to clarify or physically separate such precipitated material from that portion of the processed pellet that will be assayed with any such mycobacteriophage assay are important adjuncts to protein, enzyme and/or chemical treatments. Methods involving the use of inert matrices in the form of beads or fibers would include those products involved in chromatographic methods, such as for example, gel filtration beads, or ion exchange resins. Examples of gel filtration medias useful in the methods of the invention are the Sephadex® products, which are dextran polymers cross linked with epichlorohydrin. Cellulose powders by Whatman (United Kingdom), such as the CC31, CC41, CF1, and CF11 powders, and most especially the CDR (cell debris remover) powder are all useful in the methods of the invention. Anion exchange resins useful in the methods of the invention are those wherein cellulose beads or fibers are modified with diethylaminoethyl (DEAE) tertiary amine groups. Examples of such anion exchange resins manufactured by Whatman would include DE23, DE32, DE51, DE52, DE53, and QA52. Cation exchange resins useful in the methods of the invention are those wherein cellulose beads or fibers are modified with carboxylate or phosphate groups. Examples of such cation exchange resins manufactured by Whatman, and modified with carboxylic acids would include CM23, CM32, CM52, and examples of such cation exchange resins modified with phosphates would include P1 and P11. There are numerous gel filtration, cellulose, and ion exchange resin equivalents that are commercially available. All would be expected to be useful in the methods of the invention.

As exemplified in Example 6, such inert beads or fibers can be employed in a simple format when used in conjunction with centrifugation tubes containing adapters that have been modified to include a frit of approximately 5 microns to approximately 60 microns. Such centrifugation tubes useful in the methods of the invention can be designed to collect approximately 0.5 ml, such as the VectaSpin Micro tubes (Whatman), the Spin-X® tubes by Corning Life Sciences (U.S.A.), or the Nanosep® devices by Pall Gelman (U.S.A.). Larger versions of such centrifuge tubes designed to collect approximately 2-4 ml, and useful in the methods of the invention, would be the VectaSpin 3™ tubes (Whatman), or the Microsep™ tubes (Pall Gelman). Examples of even larger versions of such centrifuge tubes useful in the methods of the invention, (e.g., those designed to collect approximately 15-20 ml) would include the VectaSpin 20™ tubes (Whatman), or the Macrosep® tubes (Pall Gelman). There are several centrifugation tube equivalents that are commercially available. All would be expected to be useful in the methods of the invention.

Betaines useful in conjunction with the methods of the invention include the sulfobetaines and carboxybetaines, for example, the highly purified (e.g., research grade) “SB”-series of detergents. Examples of carboxybetaines useful in the methods of the invention that utilize a methylene bridge (“carboxymethylbetaines”: R₄=—CH₂—), a methylene linkage (α=—CH₂—), and vary solely based on alkyl chain length are: C₁₀ (CAS®No. 2644-45-3), C₁₁ (CAS®No. 2956-38-9), C₁₂ (CAS®No. 683-10-3), C₁₃ (CAS®No. 23609-76-9), C₁₄ (CAS®No. 2601-33-4), C₁₅ (CAS®No. 23609-77-0), C₁₆ (CAS®No. 693-33-4), and C₁₈ (CAS®No. 820-66-6). There is a C₁₂-carboxymethylbetaine (CAS®No. 6232-16-2) example that is N,N diethyl (R₃=R₄=—CH₂CH₃); and an example in which the alkyl has a double bond: C_{18:1} (CAS®No. 871-37-4). There are several carboxymethylbetaine examples in this subset in which α is an amidopropyl group. They include: C₁₂-amido (CAS®No. 4292-10-8), C₁₄-amido (CAS®No. 59272-84-3), C₁₆-amido (CAS®No. 32954-43-1), and C₁₈-amido (CAS®No. 6179-44-8). The C₁₈-amido (CAS®No. 6179-44-8) is of undefined structure because the alkyl is the “iso” form, which suggests that it branches in some undefined manner. There are several amidopropyl carboxymethylbetaines in which the alkyl chain is derived from coconut oil, and differences are due to the method of preparation. Two examples in this category include CAS®Numbers 61789-39-7 and 61789-40-0. An example of cococarboxymethylbetaine would be CAS®No. 68424-94-2. Other natural oil carboxymethyl derivatives include: ricinamidopropyl carboxymethylbetaine (CAS®No. 71850-81-2), and Tallow bishydroxyethyl glycinate (CAS®No. 70750-46-8). There are also several carboxymethylbetaines that have been tested for which no CAS®Number has been given. These include: wheat germ oil-amidopropyl carboxymethylbetaine (Schercotaine WOAB: Scher Chemicals, Inc., Clifton, N.J.), babassuamidopropyl carboxymethylbetaine (Croda, Inc., Parsippany, N.J.), soyamidopropyl carboxymethylbetaine (Chembetaine S: Chemron Corp., Paso Robles, Calif.), and canloamidopropyl betaine (Hetaine CLA: Heterene, Inc., Patterson, N.J.). There are several examples in which the nitrogen in the amide linkage is the quaternary nitrogen (e.g., the linkage (a) is a carbonyl). These include: C₁₁ (CAS®No. 66451-67-0), C₁₁ (CAS®No. 66516-99-2), and C₁₇ (CAS®No. 66451-68-1). Examples of carboxybetaines that utilize an ethyl bridge (“carboxyethylbetaine”: R₄=—CH₂CH₂—), a methylene linkage (α=—CH₂—), and vary solely based on alkyl chain length include: C₁₂ (CAS®No. 16527-85-8), C₁₃ (CAS®No. 132621-79-5), C₁₄ (CAS®No. 69725-38-3), C₁₆ (CAS®No. 42416-43-3), and C₁₈ (CAS®No. 30612-73-8). An example of a carboxyethylbetaine in which R₂ and R₃ are hydrogen atoms, under the appropriate conditions, would be CAS®No. 1462-54-0 (C₁₂-beta alanine). Examples of carboxy betaines that utilize a propyl bridge (“carboxypropylbetaine”: R₄=—CH₂CH₂CH₂—), a methylene linkage (α=—CH₂—), and vary solely based on alkyl chain length include: C₁₁ (CAS®No. 150147-53-8), C₁₂ (CAS®No. 15163-30-1), C₁₄ (CAS®No. 146959-90-2), C₁₅ (CAS®No. 146959-91-3), C₁₆ (CAS®No. 71695-32-4), and C₁₈ (CAS®No. 78195-27-4). An example of a carboxybetaine that utilizes a butyl bridge (“carboxybutylbetaine”: R₄=—CH₂CH₂CH₂CH₂—), and a methylene linkage (α=—CH₂—), would be: C₁₂ (CAS®No. 120139-51-7). Two examples of carboxy betaines that utilize a pentyl bridge (“carboxypentylbetaine”: R₄=—CH₂CH₂CH₂CH₂CH₂—), and a methylene linkage (α=—CH₂—), would be: C₁₂ (CAS®No. 76392-97-7), and C₁₆ (CAS®No. 73565-98-7). An example of a carboxy betaine that utilizes a hexyl bridge (“carboxyhexylbetaine”: R₄=—CH₂CH₂CH₂CH₂CH₂CH₂—), and a methylene linkage (α=—H₂—), would be: C₁₂ (CAS®No. 132621-80-8). There are several carboxybetaine examples in which a benzyl group is used as the bridge function (R₄=—CH₂—C₆H₄—). There are two C₁₂ examples, one in which the carboxy function is in the 4, or para, position (CAS®No. 71695-31-3), and one in which the carboxy function is in the 2, or ortho, position (CAS®No. 71695-34-6). There are two C₁₆ examples, one in which the carboxy function is in the 4, or para, position (CAS®No. 71695-33-5), and one in which the carboxy function is in the 2, or ortho, position (CAS®No. 71695-35-7). Therefore, “carboxybetaine-like” (“CB-like”) shall include those betaine-like structures that utilize a carboxyl group as the anion (γ=—COO^{⊖}), as shown in Table 1, and shall include all possible combinations of R₁, α, R₂, R₃, β, and R₄, as hereinbefore defined.

Most commercially available betaines are used to manufacture detergents, shampoos, cosmetics, and other toiletries. These betaines are derived primarily from natural oils such as coconut oil, tallow, wheat germ, babassu oil, castor oil, canola oil, soy bean oil, and rapeseed oil. The most common of these betaines includes cocoamidopropyl hydroxypropylsulfobetaine (CAS®No. 68139-30-0), cocoamidopropyl carboxymethylbetaine (CAS®No. 61789-37-9 and CAS®No. 61789-40-0), and cococarboxymethylbetaine (CAS®No. 68424-94-2). All these betaine-like detergents are useful in conjunction with the methods of the invention.

There are a variety of compounds having various chemical structures that can be used as Good buffers in the methods of the invention. In general, any chemical compound that is capable of withstanding changes in hydronium ion concentrations, and are compatible with the betaine-like detergents and a mycobacteriophage assay can be used in the methods of the invention. While small differences may appear with respect to the published or stated pKa of a particular compound, Good buffers are generally used within one pH unit of the pKa. Compounds useful as Good buffers in the methods of the inventions that are based on carboxylic acids would include: malic acid (CAS®No. 110-16-7), which has a pKa=2.0, is also known as maleate; benzoic acid (CAS®No. 65-85-0), which has a pKa=4.2, is also known as benzoate; formic acid (CAS®No. 64-18-6), which has a pKa=3.75, is also known as formate; propionic acid (CAS®No. 79-09-4), which has a pKa=4.6, is also known as propionate; acetic acid (CAS®No. 127-09-3), which has a pKa=4.76, is also known as acetate; N-[Tris(hydroxymethyl)methyl]glycine (CAS®No. 5704-04-1), which has a pKa=8.05, is also known as tricine; N,N-Bis(2-hydroxyethyl)glycine (CAS®No. 150-25-4), which has a pKa=8.26, is also known as bicine; N-(2-Acetamido)iminodiacetic acid (CAS®No. 26239-5-4), which has a pKa=6.59, is also known as ADA.

Further examples of buffers useful in the methods of the invention are those based on alcohol-substituted sulfonic acids would include: N,N-Bis(2-hydroxyethyl)-2-aminoethanesulfonic acid (CAS®No. 10191-18-1), which has a pKa=7.09, is also known as BES; N-[Tris(hydroxymethyl)methyl]-2-aminoethanesulfonic acid (CAS®No. 7365-44-8), which has a pKa=7.40, is also known as TES; N,N-Bis(2-hydroxyethyl)-3-amino-2-hydroxypropanesulfonic acid (CAS®No. 68399-80-4), which has a pKa=7.60, is also known as DIPSO; N-[Tris(hydroxymethyl)methyl]-3-amino-2-hydroxypropanesulfonic acid (CAS®No. 68399-81-5), which has a pKa=7.60, is also known as TAPSO; N-(1,1-Dimethyl-2-hydroxyethyl)-3-amino-2-hydroxypropane-sulfonic acid (CAS®No. 68399-79-1), which has a pKa=9.00, is also known as AMPSO; N-[Tris(hydroxymethyl)methyl]-3-aminopropanesulfonic acid (CAS®No. 29915-38-6), which has a pKa=8.40, is also known as TAPS; N-tris(Hydroxymethyl)methyl-4-aminobutanesulfonic acid (CAS®No. 54960-65-5), which has a pKa=8.90, is also known as TABS; N-(2-Acetamido)-2-aminoethanesulfonic acid (CAS®No. 7365-82-4), which has a pKa=6.78, is also known as ACES.

Examples of sulfonic acid compounds that would be useful as buffers in the methods of the invention are those based on morpholino-substituted compounds, for example, 2-(N-Morpholino)ethanesulfonic acid (CAS®No. 4432-31-9), which has a pKa=6.10, is also known as MES; 3-(N-morpholino)propanesulfonic acid (CAS®No. 1132-61-2), which has a pKa=7.20, is also known as MOPS; 4-(N-Morpholino)butanesulfonic acid (CAS®No. 115724-21-5), which has a pKa=7.60, is also known as MOBS; 3-Morpholino-2-hydroxypropanesulfonic acid (CAS®No. 68399-77-9), which has a pKa=6.90, is also known as MOPSO.

There are also several sulfonic acid compounds that are diethylenediamine (i.e., piperazine)-substituted that would be useful as buffers in the methods of the invention; these would include: piperazine-1,4-bis(2-ethanesulfonic acid) (CAS®No. 5625-37-6), which has a pKa=6.76, is also known as PIPES; 4-(2-Hydroxyethyl)piperazine-1-(2-hydroxypropanesulfonic acid) monohydrate (CAS®No. 68399-78-0), which has a pKa=7.80, is also known as HEPPSO, piperazine-N,N′-bis(2-hydroxypropanesulfonic acid) (CAS®No. 68189-43-5), which has a pKa=7.80, is also known as POPSO; 4-(2-Hydroxyethyl)piperazine-1-propanesulfonic acid (CAS®No. 16052-06-5), which has a pKa=8.00, is also known as EPPS; 4-(2-Hydroxyethyl)piperazine-1-ethanesulfonic acid (CAS®No. 7365-45-9), which has a pKa=7.48, is also known as HEPES; and N-(2-Hydroxyethyl)piperazine-N′-(4-butanesulfonic acid) (CAS®No. 161308-36-7), which has a pKa=8.30, is also known as HEPBS.

Another group of modified sulfonic acids that would be useful as buffers in the methods of the invention would be those that are substituted with a cyclohexyl moiety; these would include: 3-(Cyclohexylamino)-2-hydroxy-1-propanesulfonic acid (CAS®No. 73463-39-5), which has a pKa=9.60, is also known as CAPSO; 2-(Cyclohexylamino)ethanesulfonic acid (CAS®No. 103-47-9), which has a pKa=9.49, is also known as CHES; 3-(Cyclohexylamino)-1-propanesulfonic acid (CAS®No. 1135-40-6), which has a pKa=10.40, is also known as CAPS; and 4-(Cyclohexylamino)-1-butanesulfonic acid (CAS®No. 161308-34-5), which has a pKa=10.70, is also known as CABS.

Examples of alcohol-substituted amines compounds that would be useful as buffers in the methods of the invention; these would include: 2-amino-2-(hydroxymethyl)-1,3-propanediol (CAS®No. 77-86-1), which has a pKa=8.06, is also known as TRIS or TRIZMA®; 2-Amino-2-methyl-1-propanol (CAS®No. 124-68-5), which has a pKa=9.70, is also known as AMP; 2-Bis(2-hydroxyethyl)amino-2-(hydroxymethyl)-1,3-propanediol (CAS®No. 6976-37-0), which has a pKa=6.50, is also known as BIS-TRIS; 1,3-Bis[tris(hydroxymethyl)methylamino]propane (CAS®No. 64431-96-5), which has a pKa₁=6.8, and a pKa₂=9.0, is also known as Bis-Tris Propane; and 2-Amino-2-methyl-1,3-propanediol (CAS®No. 115-69-5), which has a pKa=8.80, is also known as AMPD.

The composition and method of the invention are useful for the preparation of any sample suspected of containing a microorganism having mycolic acid structures in its outer membrane. Examples of such microorganisms include microorganisms having corynomycolic acid in their outer membrane (such as, for example, Corynebacterium diphtheria); microorganisms having nocardomycolic acid in their outer membrane (for example, Nocardia asteroides); and microorganisms having mycolic acid in their outer membrane (for example, Mycobacterium tuberculosis) (see also Funke, G. et al., Clin. Micro. Rev. 10:125-159 (1997) for further discussions on coryneform bacteria (incorporated herein by reference)).

The composition and method are useful for the preparation of a sample to be assayed for the presence of any desired Mycobacterium group or complex or Mycobacterium species. Examples of such members of the Mycobacterium species include a mycobacterium complex such as M. tuberculosis (MTB) complex, M. avium (MAC) complex, MAIS complex and M. fortuitum complex, as well as fast growing and slow growing mycobacteria including specified and unspecified photochromogens, nonphotochromogens, scotochromogens, and especially M. africanum, M. asiaticum, M. avium, M. bovis, M. bovis (BCG), M. butyricum, M. chelonae, M. duvalii, M. flavescens, M. fortuitum, M. gastri, M. gordonae, M. haemophilum, M. intracellulare, M. kansasii, M. leprae, M. lepraemurium, M. linda, M. lufu, M. marinum, M. malmoense, M. microti, M. mucoscum, M. nonchromogenicum, M. paratuberculosis, M. peregrinum, M. phlei, M. rhodochrous, M. scrofulaceum, M. shimoidei, M. simiae, M. smegmatis, M. szulgai, M. terrae, M. thermoresistible, M. triviale, M. tuberculosis, M. ulcerans, M. vaccae, M. xenopi, and serovars thereof.

M. kansasii, M. marinum, M. simiae and M. asiaticum are examples of photochromogens. M. scrofulaceum, M. szulgai, M. xenopi, M. gordonae and M. flavescens are examples of scotochromogens. M. avium, M. intracellulare, M. gastri, M. malmoense, M. terrae and M. triviale are all examples of nonphotochromogens.

M. africanum, M. avium, M. bovis, M. haemophilum, M. intracellulare, M. kansasii, M. malmoense, M. marinum, M. microti, M. paratuberculosis, M. scrofulaceum, M. simiae, M. szulgai, M. tuberculosis, and M. xenopi are all examples of slow-growing (requiring more than seven days) mycobacterial species. M. chelonei, M. flavescens, M. fortuitum, M. gordonae, M. leprae, M. neoaurum, M. phlei, M. smegmatis, M. terrae, and M. ulcerans are all examples of rapid-growing (requiring less than seven days) mycobacterial species.

M. tuberculosis, M. africanum, M. bovis, M. bovis (BCG), and M. microti are the members of the Mycobacterium tuberculosis complex (MTB). M. avium and M. intracellulare are the members of the Mycobacterium avium complex (MAC); there are at least three distinct serologic groups of M. avium, and more than 25 serovars of M. intracellulare.

Examples of the diseases and conditions in which the various mycobacterial species are of heightened importance in testing include especially the causative agents of tuberculosis (M. tuberculosis complex) and leprosy (M. leprae (human leprosy) and M. lepraemurium (rodent leprosy)). Mycobacterium avium complex bacteria are important bird pathogens. M. avium has also been isolated from AIDS patients who are afflicted with a mycobacterial superinfection (Nightingale, S. D. et al., Jour. Infect. Dis. 165:1082-1085 (1992)). M. bovis is of importance in veterinary medicine. M. fortuitum is a soil bacterium that has been isolated from lesions in animals and humans. M. intracellulare is opportunistic and is especially seen in patients infected with the AIDS virus. M. paratuberculosis is of interest in the diagnosis of Crohn's disease (regional ileitis) in humans. Mycobacterium kansasii is a rare but devastating agent, generally associated with pulmonary disease. Mycobacterium marinum infects cold-blooded animals and fish; it has also been isolated from superficial granulomas on the extremities of humans. Mycobacterium paratuberculosis is the causative agent of Johne's disease in cattle; it is very slow growing and cultures must be held for 16 weeks before it can be assured that they are negative. M. ulcerans is also of interest in human medicine. Many of the above and others have been discussed by Wayne, L. G. et al., Clin. Micro. Rev. 5:1-25 (1992), and Falkinham, O. Clin. Micro. Rev. 9:177-215 (1996) and are incorporated herein by reference.

Detecting the presence of organisms containing mycolic acid structures in their outer membranes in the sample prepared by the method of the invention can be accomplished using any of the mycobacteriophage kits manufactured by Biotec Laboratories Ltd. (Ipswich, Suffolk, U.K.). The FASTPlaqueTB™ or the FASTPlaqueTB-MDRi™ kits, for example. Other variations of the plaque assay wherein colorimetric detection using β-galactosidase, or chemiluminescent detection using luciferase (the so-called “Bronx-Box”, for example (Hazbon, et al., Jour Clin. Microbiol. 41:4865-4869 (2003))) are viable means for detection microorganisms with mycolic acid structures following application of the method of the invention.

Having now fully described the invention, it will be understood by those with skill in the art that the invention may be performed within a wide and equivalent range of conditions, parameters and the like, without affecting the spirit or scope of the invention or any embodiment thereof. All references cited herein are fully incorporated herein by reference.

### EXAMPLES

### Example 1

### Exposure of Mycobacterium tuberculosis ATCC 27294 to CB-18

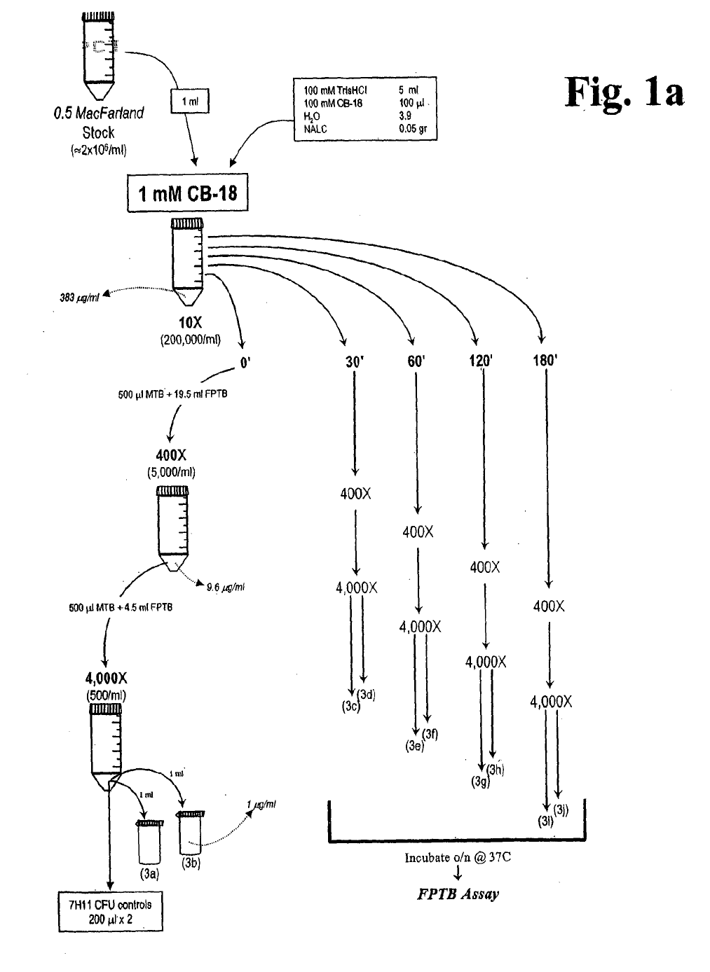

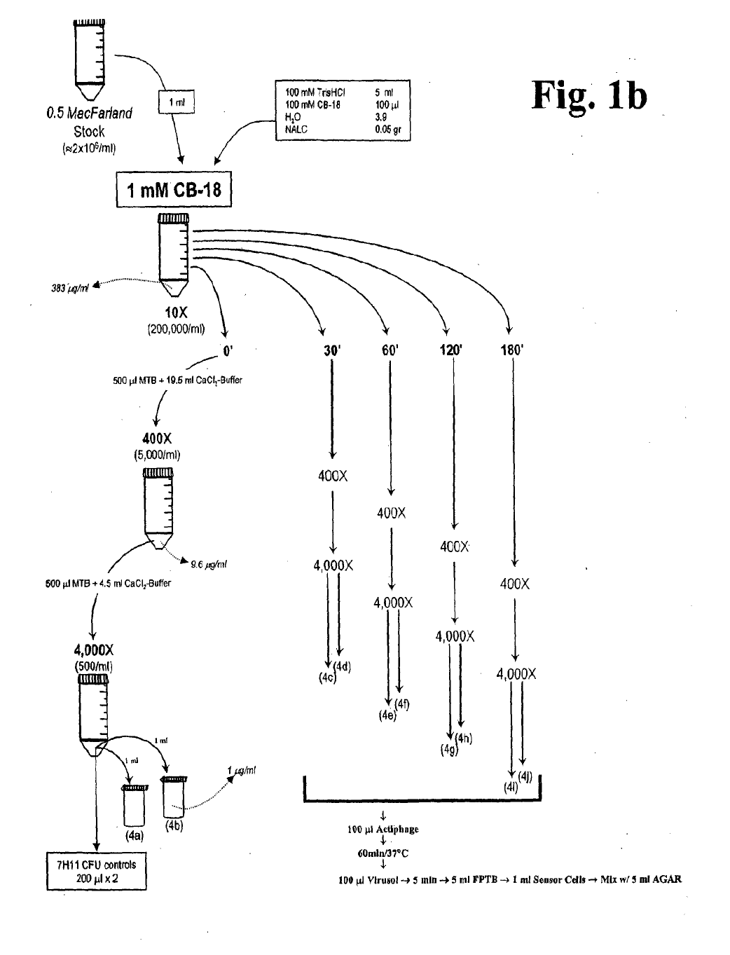

In an effort to establish the effect of exposure to CB-18 on the M. tuberculosis type strain ATCC 27294, and any relationship of such an effect to time, the following experiment was performed using the FASTPlaqueTB™ (FPTB) assay manufactured by Biotec Laboratories Ltd (Ipswich, Suffolk, U.K.): A 0.5 MacFarland stock of M. tuberculosis was manufactured as described by Thornton, C. G., et al, Jour. Clin. Microbiol. 36:2004-2013 (1998), and a 1 ml portion were transferred to a 50 ml conical tubes containing 50 mM Tris-HCl pH 7.5 @ 25° C., 66 mM NaCl, 1 mM CB-18, and 0.025% N-acetyl-L-cysteine (NALC). From this tube was taken duplicate 500 μl aliquots at 0, 30, 60, 120, and 180 minutes and immediately serially diluted 400-fold to form two dilution series. In the first series (FIG. 1a), serial dilutions were made in nutrient broth (i.e., FPTB broth) provided by the manufacturer. In the second series (FIG. 1b), serial dilutions were made in a 50 mM Tris-HCl pH 7.5 @ 25° C., 66 mM NaCl buffer that had been supplemented with calcium chloride (CaCl₂) to a final concentration of 2 mM.

The first series of time point-dilutions were then incubated at 37° C. overnight in FPTB broth prior to being assayed with the FPTB plaque assay. The second series of time point-dilutions were immediately subjected to analysis using the FPTB plaque assay. The results are shown in Table 2.

TABLE 2

| | | |
|---|---|---|
| | Plaque formation | Plaque formation |
| | following infection | following overnight |
| Time of | in Tris buffer and | pre-culture in FPTB |
| exposure | no pre-culture | nutrient broth |

| (min) | a | b | a | b |
|---|---|---|---|---|
| 0′ | 132 | 121 | >300 | >300 |
| 30′ | 145 | 167 | >300 | >300 |
| 60′ | 100 | 90 | >300 | >300 |
| 120′ | 155 | 140 | >300 | >300 |
| 180′ | 213 | 221 | >300 | >300 |

This experiment was performed in such a manner as to allow the effects associated with exposure to CB-18 to be separated from effects associated with the presence of CB-18. Due to the inherent error within the assay, and the lack of replicates, it is impossible to conclude whether there were statistically significant differences at the different time points when pre-culture was eliminated. When these results were compared to the pre-culture results, pre-culture appeared to provide more robust results. However, given that all bacilli in this experiment were exposed to CB-18, and further given that there were almost certainly bacilli in various states of replication when the 0.5 MacFarland stock was generated, it is impossible to determine whether the higher plaque numbers were due to recovery of bacilli and/or production of phage receptors, or whether the higher plaque numbers simply resulted from an in vitro amplification of bacilli by culture. Regardless, the results clearly indicate that no pre-culture step is required following exposure to CB-18.

### Example 2

### The Effect of the Presence of CB-18 on Mycobacterium tuberculosis ATCC 27294 During Infection

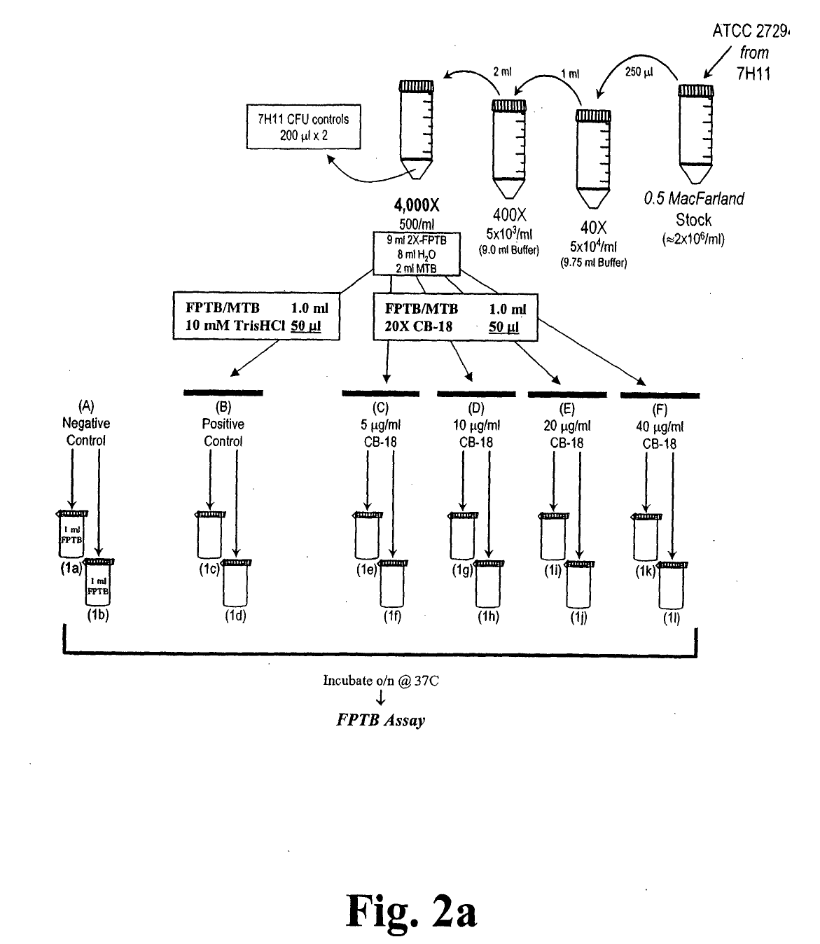

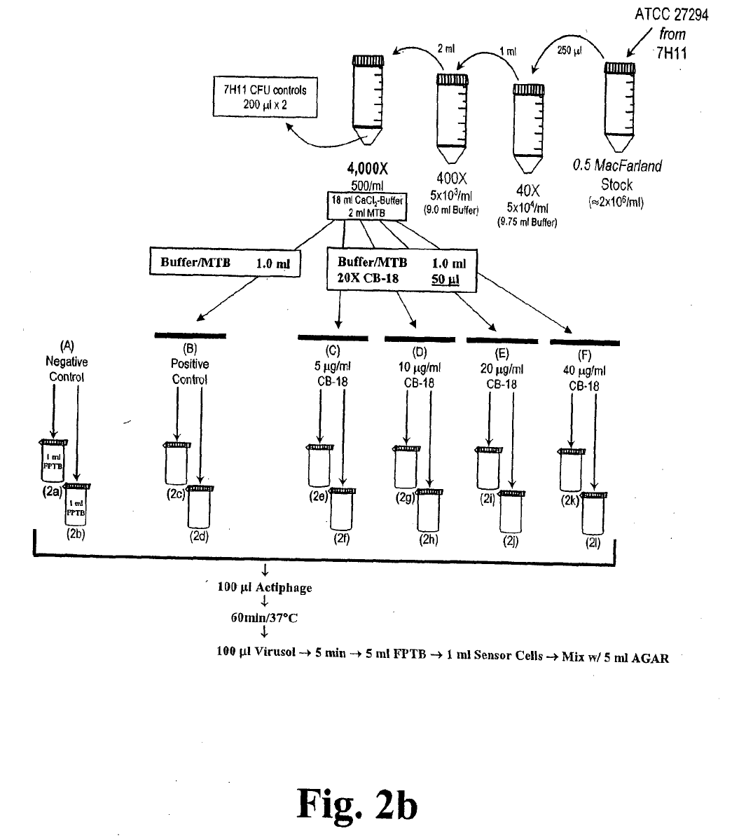

In an effort to establish the effect that the presence of CB-18 might have on the FPTB assay the following experiment was performed: A 0.5 MacFarland stock of M. tuberculosis ATCC 27294 was manufactured as described by Thornton, C. G., et al, Jour. Clin. Microbiol. 36:2004-2013 (1998). This stock was serially diluted 4.000-fold into FPTB nutrient broth (FIG. 2a). Duplicate FPTB assays were prepared and 1 ml of the diluted stock was placed in each assay tube. FPTB assay tubes were spiked with CB-18 to a final concentration of 0, 5, 10, 20 and 40 μg/ml. All tubes were then incubated overnight at 37° C. prior to detection sing the FPTB plaque assay.

In a separate experiment the 0.5 MacFarland stock was serially diluted 4,000-fold into the 50 mM Tris-HCl pH 7.5® 25° C., 66 mM NaCl, 2 mM CaCl₂ buffer (FIG. 2b). Duplicate FPTB assays were prepared and 1 ml of the diluted stock was placed in each assay tube (Table 3). FPTB assay tubes were again spiked with CB-18 to a final concentration of 0, 5, 10, 20 and 40 μg/ml. All tubes were then immediately subjected to the FPTB plaque assay.

TABLE 3

| | | |
|---|---|---|
| | Plaque formation | Plaque formation |
| | following infection | following overnight |
| Conc. of | in Tris buffer and | pre-culture in FPTB |
| CB-18 | no pre-culture | nutrient broth |

| (μg/ml) | a | b | a | b |
|---|---|---|---|---|
| 0 | 211 | 229 | >300 | >300 |
| 5 | 171 | 191 | >300 | >300 |
| 10 | 204 | 186 | >300 | >300 |
| 20 | 181 | 198 | >300 | >300 |
| 40 | 0 | 7 | >300 | >300 |

This experiment was designed to establish the need to eliminate CB-18 prior to infection. Again, when these results were compared to the pre-culture results, pre-culture appeared to provide more robust results, even at 40 μg/ml. Again, while it is impossible to determine whether the higher plaque numbers were due to recovery bacilli and/or production of phage receptors, or whether the higher plaque numbers simply resulted from an in vitro amplification of bacilli by culture, the difference in the plaque numbers between the positive controls in the two arms of the experiment suggest that the higher plaque numbers following pre-culture were due to in vitro culture amplification because neither control condition was treated or exposed to CB-18 at any point in the experiment. Regardless, the results clearly indicate that as long as CB-18 sample processing can be performed in such a way as to reduce the CB-18 carried into the infection buffer, no subsequent wash step is required.

### Example 3

### Examining the Effect of Combining Exposure to CB-18, and Carrying CB-18 into the Infection Buffer

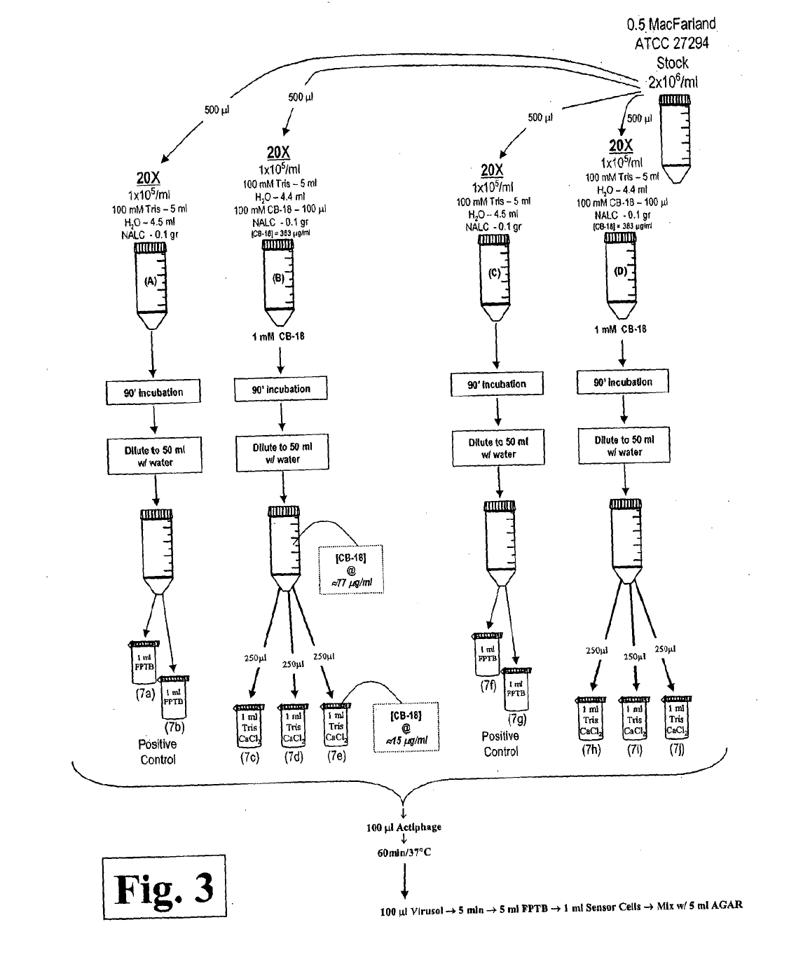

To examine the effects of both exposing M. tuberculosis to CB-18, and having CB-18 carried over into the infection buffer, the following experiment was performed: A 0.5 MacFarland stock of M. tuberculosis was manufactured as described by Thornton, C. G., et al, Jour. Clin. Microbiol. 36:2004-2013 (1998), and a 0.5 ml portions were transferred to quadruplicate 50 ml conical tubes (FIG. 3). One pair of tubes simply contained 50 mM Tris-HCl pH 7.5 @ 25° C., 66 mM NaCl, and 0.025% NALC, while the other pair of tubes contained the same buffer supplemented with 1 mM CB-18. All tubes were incubated for 90 minutes at 37° C. and then diluted with sterile water to a final volume of 50 ml. In those tubes without CB-18 present, duplicate 250 μl portions were added directly to 1 ml of FPTB nutrient broth. From those tubes that contained CB-18, triplicate 250 μl portions were added to 50 mM Tris-HCl pH 7.5 @ 25° C., 66 mM NaCl, 2 mM CaCl₂ buffer. All tubes were then immediately subjected to analysis using the FPTB plaque assay (i.e., the plaque assay was performed without pre-culture). The results are shown in Table 4.

TABLE 4

| | Plaque formation | | Plaque formation | |
|---|---|---|---|---|
| | following processing in | | following processing in | |
| | Tris buffer, no pre- | | CB-18, no pre-culture, | |
| | culture, and infection in | | and infection in Tris- | |
| | FPTB nutrient broth | | CaCl₂ buffer | |
| Replicate | a | b | a | b |
| 1 | >300 | >300 | >300 | >300 |
| 2 | >300 | >300 | >300 | >300 |
| 3 | | | >300 | >300 |

The results confirm the hypothesis that CB-18-based sample processing can be used in a very simple format wherein both the secondary wash step and the pre-culture step can be eliminated if the amount of CB-18 carried over into the infection buffer can be kept below 20 μg/ml.

### Example 4

### The Effect of exposure of Mycobacterium tuberculosis ATCC 27294 to Lytic Enzymes before Infection

To establish the effect of exposure of M. tuberculosis type strain ATCC 27294, to lytic enzymes, the following experiment was performed using the FASTPlaqueTB™ (FPTB) assay manufactured by Biotec Laboratories Ltd (Ipswich, Suffolk, U.K.): A composition of lytic enzymes consisting of lysozyme (10 mg/ml), Trichoderma extract (15 units β-glucanase), and Lysobacter extract (10 units of chitinase/ml) was made as a 10-fold concentrate in 100 mM Tris-HCl pH 7.5 @25° C., 66 mM NaCl, and frozen at −20° C. in 1 ml aliquots until use. Just prior to use, an aliquot was thawed, diluted with 4 ml sterile water, and then NALC was to 0.05%.

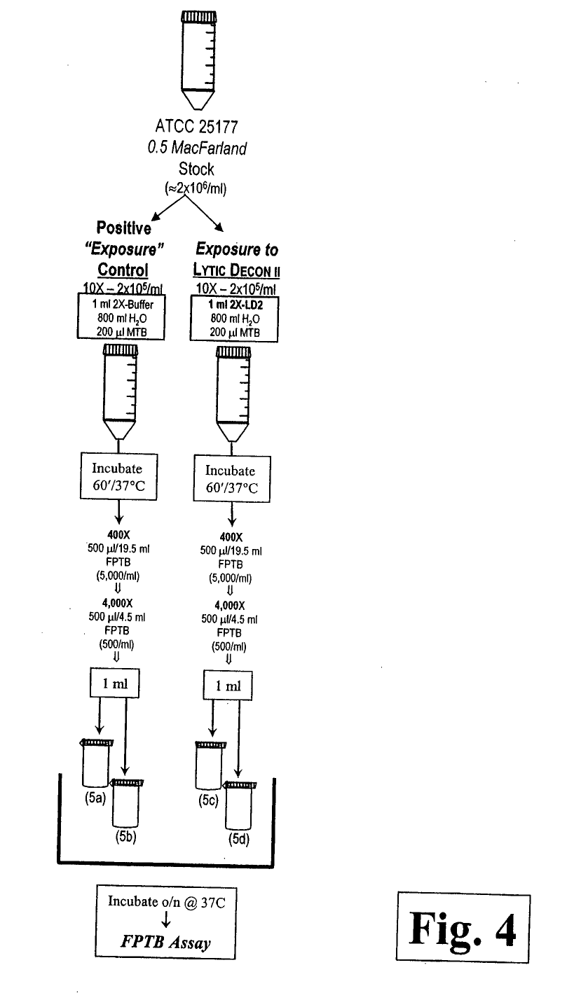

A 0.5 MacFarland stock of M. tuberculosis was manufactured as described by Thornton, C. G., et al, Jour. Clin. Microbiol. 36:2004-2013 (1998), and 200 μl portions were transferred to two 50 ml conical tubes (FIG. 4). One tube contained 50 mM Tris-HCl pH 7.5 @ 25° C., 66 mM NaCl, and 0.05% NALC (i.e., the control), while the other tube contained the fully diluted (i.e., 1-fold) enzyme mixture. Both tubes were incubated at 37° C. for 60 minutes. At the end of the incubation period a 500 μl aliquot from each tube was serially diluted 400-fold into FPTB nutrient broth. Duplicate 1 ml portions of each 4,000-fold dilution were assayed.

TABLE 5

| | a | b |
|---|---|---|
| Control | >300 | >300 |
| Lytic enzymes | >300 | >300 |

These results clearly indicate that M. tuberculosis can be exposed to lytic enzymes prior to infection (Table 5).

### Example 5

### Processing Clinical Specimens and Inhibition of the FASTPlaqueTB™ Assay

The impact of actual clinical specimens on the performance of the FASTPlaqueTB assay was examined. Eight random, discarded respiratory specimens were collected from the Microbiology Department at Quest Diagnostics in Baltimore, Md. To each specimen was added 5 ml of CB-18 processing buffer (100 mM Tris-HCl, pH 7.5 @ 25° C., 66 mM NaCl, 2 mM CB-18, and 0.5% NALC). Specimens were incubated for 90 minutes at room temperature, diluted with sterile water to 50 ml, and then subjected to centrifugation for 20 minutes at 3,000×g at 25° C. All sediments were resuspended in 2 ml of sterile water. From each sediment, a 900 μl aliquot was taken and mixed with approximately 2,000 CFU of M. tuberculosis ATCC 27294. The remaining sediment was washed with 20 ml of FPTB broth, and subjected to a second centrifugation for 20 minutes at 3,000×g at 25° C. The washed sediment was resuspended in 1 ml of sterile water, and again a 900 μl aliquot was taken and mixed with approximately 2,000 CFU of M. tuberculosis ATCC 27294. From each pair of sediments (primary [1°] sediment and washed sediment), a 250 μl aliquot was taken and mixed with 1 ml of infection buffer (50 mM Tris-HCl pH 7.5 @ 25° C., 33 mM NaCl, 2 mM CaCl₂). The FASTPlaqueTB™ assay was then carried out as per the manufacturer's instructions. The results are shown in Table 6.

TABLE 6

| | Specimen characteristics before and after | | | |
|---|---|---|---|---|
| | processing with CB-18 | | # Plaques | |
| Spec. # | Before | After | 1° Sediment | Washed |
| 1 | 1-2 ml B-W | small, white pellet | 0 | 0 |
| 2 | 2 ml B-W | small, white pellet | 0 | 0 |
| 3 | 2-3 ml sputum | small, white pellet | 1 | 1 |
| 4 | 3-4 ml sputum | medium, white pellet | 0 | 0 |
| 5 | 4-5 ml sputum | medium, white pellet | 5 | 0 |
| 6 | 5 ml purulent | heavy, white pellet | 0 | 1 |
| 7 | 7.5 ml thick mucus | heavy, thick pellet | 0 | 0 |
| 8 | 5 ml gelatinous mucus | gelatinous pellet | 0 | 0 |

| | |
|---|---|
| Positive control plate | >300 |
| Negative control plate | 0 |

Of the 16 plates testing assay performance with respiratory specimens, only 8 plaques were observed. The input into each assay (based on the positive control plate) was approximately 300-400 CFU. Therefore, of the 16 test plates, one could expect between 4,800 and 6,400 total plaques (i.e., the sum of all 16 test plates) if no inhibition were observed. Inhibition of the assay by components of the processed sediment is dramatic, and washing sediments under these conditions did not help.

The previous experiment was repeated with the exception that specimens were pretreated with an equal volume of 50 mM NaOH containing 0.5% NALC for 15 minutes to facilitate dispersion and liquefaction of specimens (FIG. 6). In addition, sediments were not washed, but instead incubated overnight in 1 ml of FPTB broth as recommended by the manufacturer to allow recovery of phage receptors following treatment with NaOH. The results are shown in Table 7.

TABLE 7

| | Specimen characteristics before and after | | |
|---|---|---|---|
| | dispersing with NaOH and processing with CB-18 | | # Plaques |
| Spec. # | Before | After | Sediment |
| 1 | 1-2 ml B-W | small, white pellet | 0 |
| 2 | 1-2 ml B-W | small, white pellet | 0 |
| 3 | 1-2 ml thin sputum | small, white pellet | contaminated |
| 4 | 2-3 ml thin sputum | small, white pellet | 0 |
| 5 | 3-4 ml thin sputum | small, white pellet | 0 |
| 6 | 1-2 ml thick sputum | heavy, white pellet | contaminated |
| 7 | 2-3 ml thick sputum | heavy, white pellet | contaminated |
| 8 | 5 ml thick sputum | heavy, white pellet | 0 |

| | |
|---|---|
| Positive control plate | >300 |
| Negative control plate | 0 |

The results again indicate the magnitude of the inhibition problem. In addition, due to the overnight incubation, several plates were contaminated to the degree that the lawn did not appear to form, suggesting that overnight incubations following CB-18 specimen processing require the presence of antibiotics.

Having now fully described the invention, it will be understood by those with skill in the art that the invention may be performed within a wide and equivalent range of conditions, parameters and the like, without affecting the spirit or scope of the invention or any embodiment thereof.

What is claimed is:

1. A method of preparing a specimen or extract thereof for use in a mycobacteriophage assay to detect the presence of a microorganism having mycolic acid structures in its outer membrane that is suspected of being present in said sample, said method comprising:
(a) treating a specimen or extract thereof with a betaine-like detergent;
(b) analyzing the treated specimen or extract thereof of part (a) for the presence of said microorganism by a mycobacteriophage assay.

2. The method of claim 1, wherein said specimen or extract thereof is not pre-cultured after said treating of part (a) and prior to said analyzing of part (b).

3. The method of claim 1, wherein said specimen or extract thereof is pre-cultured after said treating of part (a) and prior to said analyzing of part (b).

4. The method of any of claims 1-3, wherein said treating of said specimen or extract thereof of part (a) is in a buffer that is compatible with said mycobacteriophage assay.

5. The method of claim 4, wherein said buffer that is used in said treating is the same as the buffer that is used in said analyzing.

6. The method of any of claims 1-3, wherein said betaine-like detergent is selected from the group consisting of CB-like, SB-like, HSB-like, PB-like, StB-like, PhB-like, SoB-like, RevB-like, AO-like, cAB-like, and ImB-like detergents.

7. The method of claim 6, wherein said betaine-like detergent is a CB-like detergent.

8. The method of claim 7, wherein said CB-like detergent has the structure 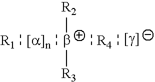
wherein R₁ is C₈-C₂₂;
α is ¦CH₂¦, ¦CH(OH) ¦, ¦(CO)—NH—CH₂CH₂CH₂¦, ¦O¦, or ¦C(O)¦;
n is 0 or 1;
β is ¦N^{⊖}¦, ¦P^{⊖}¦, or ¦S^{⊖}¦;
R₂ is ¦H, ¦CH₃, ¦C₂H₅, ¦C₃H₇, or ¦C₄H₉;
R₃ is ¦H, ¦CH₃, ¦C₂H₅, ¦C₃₁H₇, or ¦C₄H₉;
R₄ is ¦CH₂¦, ¦C₂H₄¦, ¦C₃H₆, ¦C₄H₈¦, ¦C₅H₁₀¦, ¦C₆H₁₂¦, CH₂¦C₆H₄¦, ¦CₘH₂ₘ¦, ¦CH(OH)CH₂CH₂¦, ¦CH₂CH(OH)CH₂¦, or ¦CₘH₂ₘ₋₁(OH)¦ where m is ≧1; and
γ is —COO^{⊕}.

9. The method of claim 8, wherein said CB-like detergent is selected from the group consisting of N-(carboxymethyl)-N,N-dimethyl-1-hexadecanaminium, inner salt (CAS®No. 693-33-4),
cococarboxymethylbetaine and (CAS®No. 68424-94-2),
N-(carboxymethyl)-N,N-dimethyl-9-octadecen-1-aminium, inner salt (CAS®No. 871-37-4),
N-(carboxymethyl)-N,N-dimethyl-3-((1-oxooctadecyl)amino)-1-propanaminium, inner salt (CAS®No. 6179-44-8),
3-amino-N(carboxymethyl)-N,N-dimethyl-1-propanaminium N—C₈-C₂₂ acyl derivatives, inner salt (CAS®No. 84082-44-0),
N-(carboxymethyl)-3-((12-hydroxy-1-oxo-9-octadecenyl)amino)-N,N-dimethyl-1-propanaminium, inner salt (CAS®No. 71850-81-2),
cocoamidopropyl carboxymethylbetaine (CAS®No. 61789-39-7 and CAS®No. 61789-40-0),
N-(2-carboxyethyl)-N,N-dimethyl-1-dodecanaminium, inner salt (CAS®No. 16527-85-8),
N-(2-carboxyethyl)-N,N-dimethyl-1-tridecanaminium, inner salt (CAS®No. 132621-79-5),
N-(2-carboxyethyl)-N,N-dimethyl-1-tetradecanaminium, inner salt (CAS®No. 69725-38-3),
N-(2-carboxyethyl)-N,N-dimethyl-1-hex adecanaminium, inner salt (CAS®No. 42416-43-3),
N-(2-carboxyethyl)-N,N-dimethyl-1-octadecanaminium, inner salt (CAS®No. 30612-73-8),
N-dodecyl-beta-alanine (CAS®No. 1462-54-0),
N-(3-carboxypropyl)-N,N-dimethyl-1-undecanaminium, inner salt (CAS®No. 150147-53-8),
N-(3-carboxypropyl)-N,N-dimethyl-1-dodecanaminium, inner salt (CAS®No. 15163-30-1),
N-(3-carboxypropyl)-N,N-dimethyl-1-tetradecanaminium, inner salt (CAS®No. 146959-90-2),
N-(3-carboxypropyl)-N,N-dimethyl-1-pentadecanaminium, inner salt (CAS®No. 146959-91-3),
N-(3-carboxypropyl)-N,N-dimethyl-1-hexadecanaminium, inner salt (CAS®No. 71695-32-4),
N-(3-carboxypropyl)-N,N-dimethyl-1-octadecanaminium, inner salt (CAS®No. 78195-27-4),
N-(4-carboxybutyl)-N,N-dimethyl-1-dodecanaminium, inner salt (CAS®No. 120139-51-7),
N-(5-carboxypentyl)-N,N-dimethyl-1-dodecanaminium, inner salt (CAS®No. 76392-97-7),
N-(5-carboxypentyl)-N,N-dimethyl-1-hexadecanaminium, inner salt (CAS®No. 73565-98-7),
N-(6-carboxyhexyl)-N,N-dimethyl-1-dodecanaminium, inner salt (CAS®No. 132621-80-8),
4-carboxy-N-dodecyl-N,N-dimethyl-benzenemethanaminium, inner salt (CAS®No. 71695-31-3),
2-carboxy-N-dodecyl-N,N-dimethyl-benzenemethanaminium, inner salt (CAS®No. 71695-34-6),
4-carboxy-N-hexadecyl-N,N-dimethyl-benzenemethanaminium, inner salt (CAS®No. 71695-33-5),
2-carboxy-N-hexadecyl-N,N-dimethyl-benzenemethanaminium, inner salt (CAS®No. 71695-35-7),
tallow glycinate (CAS®No. 70750-46-8),
soyamidopropyl carboxymethylbetaine, and
babassuamidopropyl carboxymethylbetaine.

10. The method of claim 9, wherein said CB-like detergent is selected from the group consisting of N-(2-carboxyethyl)-N,N-dimethyl-1-dodecanaminium, inner salt (CAS®No. 16527-85-8),
N-(2-carboxyethyl)-N,N-dimethyl-1-tridecanaminium, inner salt (CAS®No. 132621-79-5),
N-(2-carboxyethyl)-N,N-dimethyl-1-tetradecanaminium, inner salt (CAS®No. 69725-38-3),
N-(2-carboxyethyl)-N,N-dimethyl-1-hexadecanaminium, inner salt (CAS®No. 42416-43-3),
N-(2-carboxyethyl)-N,N-dimethyl-1-octadecanaminium, inner salt (CAS®No. 30612-73-8),
N-(3-carboxypropyl)-N,N-dimethyl-1-undecanaminium, inner salt (CAS®No. 150147-53-8),
N-(3-carboxypropyl)-N,N-dimethyl-1-dodecanaminium, inner salt (CAS®No. 15163-30-1),
N-(3-carboxypropyl)-N,N-dimethyl-1-tetradecanaminium, inner salt (CAS®No. 146959-90-2),
N-(3-carboxypropyl)-N,N-dimethyl-1-pentadecanaminium, inner salt (CAS®No. 146959-91-3),
N-(3-carboxypropyl)-N,N-dimethyl-1-hexadecanaminium, inner salt (CAS®No. 71695-32-4),
N-(3-carboxypropyl)-N,N-dimethyl-1-octadecanaminium, inner salt (CAS®No. 78195-27-4), and
N-(4-carboxybutyl)-N,N-dimethyl-1-dodecanaminium, inner salt (CAS®No. 120139-51-7).

11. The method of claim 10, wherein said carboxybetaine is N-(3-carboxypropyl)-N,N-dimethyl-1-octadecanaminium, inner salt (CB-18) (CAS®No. 78195-27-4).

12. The method of claim 6, wherein said betaine-like detergent is an SB-like detergent.

13. The method of claim 12, wherein said SB-like detergent is selected from the group consisting of SB-18, SB-16, SB-14 and SB-12.

14. The method of claim 13, wherein said SB-like detergent is said SB-16.

15. The method of claim 13, wherein said SB-like detergent is said SB-18.

16. The method of any one of claims 1-3, wherein the treated specimen or said treated extract thereof is not cultured after said treating of part (a) and before said analyzing of part (b).

17. The method of any one of claims 1-3, wherein said treating said specimen or extract thereof comprises treating with a composition that comprises one or more lytic enzymes that are active against the outer membranes of gram positive bacteria, gram negative bacteria or mycolic organisms.

18. The method of claim 17, wherein said lytic enzymes comprise one or more members of the group consisting of lysozyme, lyticase, Trichoderma lytic enzymes, Cytophaga lytic enzymes, Lysobacter lytic enzymes, and Micromonospora lytic enzymes.

19. The method of claim 18, wherein said composition comprises lysozyme.

20. The method of claim 18, wherein said composition comprises lyticase.

21. The method of claim 18, wherein said composition comprises Trichoderma lytic enzymes.

22. The method of claim 18, wherein said composition comprises Cytophaga lytic enzymes.

23. The method of claim 18, wherein said composition comprises Lysobacter lytic enzymes.

24. The method of claim 18, wherein said composition comprises Micromonospora lytic enzymes.

25. The method of claim 18, wherein said composition comprises lysozyme, lyticase, Trichoderma lytic enzymes and Lysobacter lytic enzymes.

26. The method of claim 18, wherein said composition comprises lysozyme, lyticase, Trichoderma lytic enzymes, Lysobacter lytic enzymes and Micromonospora lytic enzymes.

27. The method of claim 18, wherein said betaine-like detergent is added before said lytic enzymes.

28. The method of claim 18, wherein said betaine-like detergent is added after said lytic enzymes.

29. The method of claim 18, wherein said betaine-like detergent is added at the same time as said lytic enzymes.

30. The method of any of claims 1-3, wherein said method further comprises the addition of one or more antibiotics.

31. The method of any of claims 1-3, further comprising the use of mechanical disruption.

32. The method of claim 31, wherein said mechanical disruption is sonication.

33. The method of any one of claims 1-3, wherein the outer membrane of said bacteria contains mycolic acid like structures.

34. The method of claim 33, wherein said bacteria is a member of the mycobacteria.

35. The method of claim 34, wherein said member of the mycobacteria is anyone of the Mycobacterium tuberculosis complex, Mycobacterium avium complex, Mycobacterium kansasii, and or Mycobacterium fortuitum complex.

36. The method of claim 35, wherein said member of the mycobacteria is Mycobacterium tuberculosis complex.

37. The method of claim 35, wherein said member of the mycobacteria is Mycobacterium avium complex.

38. The method of claim 35, wherein said member of the mycobacteria is Mycobacterium kansasii.

39. The method of claim 35, wherein said member of the mycobacteria is Mycobacterium fortuitum complex.

40. The method of any of claims 1-3, wherein said specimen is a tissue specimen.

41. The method of any of claim 1-3, wherein said specimen is a biological fluid specimen.

42. The method of any of claims 1-3, wherein said specimen is a food.
